SEQUENCE LISTING (12 sequences)

SEQ ID NO: 1 - DNA, 35 bp, Artificial
  Synthetic construct, Oligo A
nttttttttttttttacgactacgttcaggcaagn

SEQ ID NO: 2 - DNA, 39 bp, Artificial
  Synthetic construct, Oligo B
nttttttttttttttttttacgactacgttcaggcaagn

SEQ ID NO: 3 - DNA, 45 bp, Artificial
  Synthetic construct, Oligo C
nttttttttttttttttttttttttacgactacgttcaggcaagn

SEQ ID NO: 4 - DNA, 20 bp, Artificial
  Synthetic construct, Oligo D
nacctgtcgagcatccagcn

SEQ ID NO: 5 - DNA, 11 bp, Artificial
  Synthetic construct, Oligo E
ncatccatcgn

SEQ ID NO: 6 - DNA, 46 bp, Artificial
  Synthetic construct, Oligo A2
gacctgtcgagcatccagcttcatgggaattcctcgtccacaatgn

SEQ ID NO: 7 - DNA, 20 bp, Artificial
  Synthetic construct, Oligo F
ncgtaacgactgaatgacgt

SEQ ID NO: 8 - DNA, 41 bp, Artificial
  Synthetic construct, template oligo G
acgtcattcagtcgttacgaacgatggatgctccaggtcgc

SEQ ID NO: 9 - DNA, 21 bp, Artificial
  Synthetic construct, Oligo G
gcgacctggagcatccatcgn

SEQ ID NO: 10 - DNA, 21 bp, Artificial
  Synthetic construct, Oligo H
gcgacctggagcatccatcgn

SEQ ID NO: 11 - DNA, 21 bp, Artificial
  Synthetic construct, Scaffold oligo I
ncgatggatgctccaggtcgc

SEQ ID NO: 12 - DNA, 56 bp, Artificial
  Synthetic construct, Oligo N
ngtaacacctgtgtaagctgcctgtcagtcggtactgacctgtcgagcatccagct

(19) United States
(12) Patent Application Publication
(10) Pub. No.: US 2006/0166197 A1
(43) Pub. Date: Jul. 27, 2006

(54) Building block capable of transferring a functional entity

(76) Inventors: Alex Haahr Gouliaev, Veksoe Sjaelland (DK); Justin Ho, Copenhagen V (DK); Jakob Felding, Charlottenlund (DK); Christian Sams, Frederiksberg C (DK); Henrik Pedersen, Bagsvaed (DK); Kim Birkebaek Jensen, Roedovre (DK); Anders Holm Hansen, Copenhagen NV (DK); Mikkel Dybro Lundorf, Copenhagen NV (DK); Gitte Nystrup Husemoen, Copenhagen N (DK); Thomas Franch, Copenhagen N (DK); Thomas Thisted, Frederikssund (DK)

Correspondence Address: BROWDY AND NEIMARK, P.L.L.C.;624 NINTH STREET, NW, SUITE 300, WASHINGTON, DC, 20001-5303, US

(21) Appl. No.: 10/507,842

(22) PCT Filed: Mar. 14, 2003
(86) PCT No.: PCT/DK03/00174

§ 371 Date: Jun. 23, 2005

Related U.S. Application Data
(60) Provisional application No. 60/364,056, filed on Mar. 15, 2002.
(60) Provisional application No. 60/434,427, filed on Dec. 19, 2002.

(30) Foreign Application Priority Data
Mar. 15, 2002 (DK) PA 2002 0415
Jun. 20, 2002 (DK) 02/00419
Jun. 20, 2002 (US) 10/175539

(51) Int. Cl. C40B 40/08; C07H 21/02; C07F 9/6512

(52) U.S. Cl. 435/6; 435/7.1; 536/23.1; 544/243

(57) ABSTRACT

A building block having the dual capabilities of transferring the genetic information e.g. by recognising an encoding element and transferring a functional entity to a recipient reactive group is disclosed. The building block can be designed with an adjustable transferability taking into account the components of the building block. The building block may be used in the generation of a single complex or libraries of different complexes, wherein the complex comprises an encoded molecule linked to an encoding element. Libraries of complexes are useful in the quest for pharmaceutically active compounds.

### TECHNICAL FIELD OF THE INVENTION

The present invention relates to a building block comprising a complementing element and precursor for a functional entity. The building block is designed to transfer the functional entity with an adjustable efficiency to a recipient reactive group upon recognition between the complementing element and an encoding element associated with the reactive group. The invention also relates to a linkage between the functional entity and the complementing element as well as a method for transferring a functional entity to recipient reactive group.

### BACKGROUND

The transfer of a chemical entity from one mono-, di- or oligonucleotide to another has been considered in the prior art. Thus, N. M. Chung et al. (Biochim. Biophys. Acta, 1971, 228,536-543) used a poly(U) template to catalyse the transfer of an acetyl group from 3′-O-acetyladenosine to the 5′-OH of adenosine. The reverse transfer, i.e. the transfer of the acetyl group from a 5′-O-acetyladenosine to a 3′-OH group of another adenosine, was also demonstrated.

Walder et al. Proc. Natl. Acad. Sci. USA, 1979, 76, 51-55 suggest a synthetic procedure for peptide synthesis. The synthesis involves the transfer of nascent immobilized polypeptide attached to an oligonucleotide strand to a precursor amino acid attached to an oligonucleotide. The transfer comprises the chemical attack of the amino group of the amino acid precursor on the substitution labile peptidyl ester, which in turn results in an acyl transfer. It is suggested to attach the amino acid precursor to the 5′ end of an oligonucleotide with a thiol ester linkage.

The transfer of a peptide from one oligonucleotide to another using a template is disclosed in Bruick R K et al. Chemistry & Biology, 1996, 3:49-56. The carboxy terminal of the peptide is initially converted to a thioester group and subsequently transformed to an activated thioester upon incubation with Ellman's reagent. The activated thioester is reacted with a first oligo, which is 5′-thiol-terminated, resulting in the formation of a thio-ester linked intermediate. The first oligonucleotide and a second oligonucleotide having a 3′ amino group is aligned on a template such that the thioester group and the amino group are positioned in close proximity and a reaction is effected resulting in a coupling of the peptide to the second oligonucleotide through an amide bond.

In an aspect of the present invention a storable oligonucleotide conjugated to a transferable chemical moiety is provided. In another aspect of the invention an oligonucleotide conjugate which is possible to prepare in a few steps is provided. In yet another aspect an arsenal of possibilities for adjusting the transferability of a chemical moiety is provided. Adjusting the transferability of a chemical moiety may prove crucial in obtaining specific reactions.

### SUMMARY OF THE INVENTION

The present invention relates to a building block of the general formula
Complementing Element—Linker—Carrier—C-F-connecting group—Functional Entity Precursor

capable of transferring a functional entity to a recipient reactive group, wherein

Complementing Element is a group identifying the functional entity precursor,

Linker is a chemical moiety comprising a Spacer and a S—C-connecting group, wherein the Spacer is a valence bond or a group distancing the functional entity precursor to be transferred from the complementing element and the S—C-connecting group connects the spacer with the Carrier,

Carrier is selected among the groups

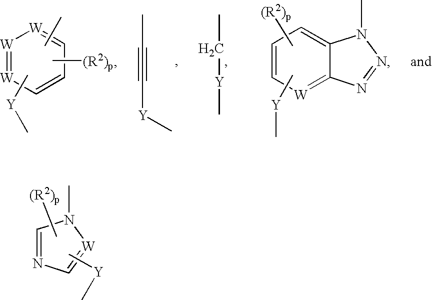

wherein the Linker attaches to the Carrier through Y and

W═CH or N

R²═—H, -Halogen, —NO₂, —CN, —C(Halogen)₃, —C(O)R³, —C(O)NHR³, C(O)NR³₂, —NC(O)R³, —S(O)₂NHR³, —S(O)₂NR³₂, —S(O)₂R³₁, —P(O)₂—R³, —P(O)—R³, —S(O)—R³, P(O)—OR³, —(O)—OR³, —N⁺R³₃, wherein p is an integer of 0 to 3, R³═H, C₁-C₆ alkyl, C₁-C₆ alkenyl, C₁-C₆ alkynyl, or aryl, and Halogen is F, Cl, Br, or I,

Y=absent, C₁-C₆ Alkylene, C₁-C₆ Alkenylene, C₁-C₆ Alkynylene, Arylene, Heteroarylene, Carbonyl, or —SO₂CH₂—,

C—F-connecting group is 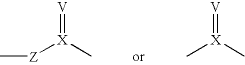
where the carrier is connected to the left hand side of the formulae and

X═—C—, —S—, —P—, —S(O)— or —P(O)—,

V═O, S, NH, or N—C₁-C₆ alkyl, and

Z=O, S, and

Functional entity precursor is H or selected among the group consisting of a C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R⁴, 0-3 R⁵ and 0-3 R⁹, or selected among the group consisting of C₁-C₃ alkylene-NR⁴₂, C₁-C₃ alkylene-NR⁴C(O)R⁸, C₁-C₃ alkylene-NR⁴C(O)OR⁸, C₁-C₂ alkylene-O—NR⁴₂, C₁-C₂ alkylene-O—NR⁴C(O)R⁸, and C₁-C₂ alkylene-O—NR⁴C(O)OR⁸ substituted with 0-3 R⁹.

where R⁴ is H or selected independently among the group consisting of C₁-C₆ alkyl, C₂-C₆ alkenyl, C₂-C₆ alkynyl, C₃-C₇ cycloalkyl, C₃-C₇ cycloheteroalkyl, aryl, heteroaryl, said group being substituted with 0-3 R⁹ and

R⁵ is selected independently from —N₃, —CNO, —C(NOH)NH₂, —NHOH, —NHNHR⁶, —C(O)R⁶, —SnR⁶₃, —B(OR⁸)₂, —P(O)(OR⁶)₂ or the group consisting of C₂-C₆ alkenyl, C₂-C₈ alkynyl, C₄-C₈ alkadienyl said group being substituted with 0-2 R⁷,

where R⁶ is selected independently from H, C₁-C₆ alkyl, C₃-C₇ cycloalkyl, aryl or C₁-C₆ alkylene-aryl substituted with 0-5 halogen atoms selected from —F, —C, —Br, and —I; and R⁷ is independently selected from —NO₂, —COOR⁶, —COR⁶, —CN, —OSiR⁶₃, —OR⁶ and —NR⁶₂. R⁸ is H, C₁-C₆ alkyl, C₂-C₆ alkenyl, C₂-C₆ alkynyl, C₃-C₇ cycloalkyl, aryl or C₁-C₆ alkylene-aryl substituted with 0-3 substituents independently selected from —F, —Cl, —NO₂, —R³, —OR³, —SiR³₃ R⁹ is ═O, —F, —Cl, —Br, —I, —CN, —NO₂, —OR⁶, —NR⁶₂, —NR⁶—C(O)R⁸, —NR⁶—C(O)OR⁸, —SR⁶, —S(O)R⁶, —S(O)₂R⁶, —COOR⁶, —C(O)NR⁶₂ and —S(O)₂NR⁶₂.

In the present description and claims, the direction of connections between the various components of a building block should be read left to right. For example an S—C-connecting group —C(═O)—NH— is connected to a Spacer through the carbon atom on the left and to a Carrier through the nitrogen atom on the right hand side.

The term “C₃-C₇ cycloheteroalkyl” as used herein refers to a radical of totally saturated heterocycle like a cyclic hydrocarbon containing one or more heteroatoms selected from nitrogen, oxygen, phosphor, boron and sulphur independently in the cycle such as pyrrolidine (1-pyrrolidine; 2-pyrrolidine; 3-pyrrolidine; 4-pyrrolidine; 5-pyrrolidine); pyrazolidine (1-pyrazolidine; 2-pyrazolidine; 3-pyrazolidine; 4-pyrazolidine; 5-pyrazolidine); imidazolidine (1-imidazolidine; 2-imidazolidine; 3-imidazolidine; 4-imidazolidine; 5-imidazolidine); thiazolidine (2-thiazolidine; 3-thiazolidine; 4-thiazolidine; 5-thiazolidine); piperidine (1-piperidine; 2-piperidine; 3-piperidine; 4-piperidine; 5-piperidine; 6-piperidine); piperazine (1-piperazine; 2-piperazine; 3-piperazine; 4-piperazine; 5-piperazine; 6-piperazine); morpholine (2-morpholine; 3-morpholine; 4-morpholine; 5-morpholine; 6-morpholine); thiomorpholine (2-thiomorpholine; 3-thiomorpholine; 4-thiomorpholine; 5-thiomorpholine; 6-thiomorpholine); 1,2-oxathiolane (3(1,2-oxathiolane); 4-(1,2-oxathiolane); 5-(1,2-oxathiolane); 1,3-dioxolane (2-(1,3-dioxolane); 4-(1,3-dioxolane); 5-(1,3-dioxolane); tetrahydropyrane; (2-tetrahydropyrane; 3-tetrahydropyrane; 4-tetrahydropyrane; 5-tetrahydropyrane; 6-tetrahydropyrane); hexahydropyridazine (1-(hexahydropyridazine); 2-(hexahydropyridazine); 3-(hexahydropyridazine); 4-(hexahydropyridazine); 5-(hexahydropyridazine); 6-(hexahydropyridazine)), [1,3,2]dioxaborolane, [1,3,6,2]dioxazaborocane

The term “aryl” as used herein includes carbocyclic aromatic ring systems of 5-7 carbon atoms. Aryl is also intended to include the partially hydrogenated derivatives of the carbocyclic systems as well as up to four fused aromatic- or partially hydrogenated rings, each ring comprising 5-7 carbon atoms.

The term “heteroaryl” as used herein includes heterocyclic unsaturated ring systems containing, in addition to 2-18 carbon atoms, one or more heteroatoms selected from nitrogen, oxygen and sulphur such as furyl, thienyl, pyrrolyl, heteroaryl is also intended to include the partially hydrogenated derivatives of the heterocyclic systems enumerated below.

The terms “aryl” and “heteroaryl” as used herein refers to an aryl which can be optionally substituted or a heteroaryl which can be optionally substituted and includes phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazolyl, 1,2,4-triazol-3-yl), oxazolyl (2-oxazolyl, 4oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2quinolyl, 3-quinolyl, 4quinolyl, 5-quinolyl, 6-quinolyl, 7quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3dihydro-benzo[b]thiophenyl (2-(2,3-dihydro-benzo[b]thiophenyl), 3-(2,3-dihydro-benzo[b]thiophenyl), 4-(2,3dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3yl, 5H-dibenz[b,f]azepine-4-yl, 5H-dibenz[b,f]azepine-5-yl), 10,11 -dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine4-yl, 10,11-dihydro-5H-dibenz[b,f]azepine-5-yl).

The Functional Entity carries elements used to interact with host molecules and optionally reactive elements allowing further elaboration of an encoded molecule of a library. Interaction with host molecules like enzymes, receptors and polymers is typically mediated through van der waal's interactions, polar- and ionic interactions and pi-stacking effects. Substituents mediating said effects may be masked by methods known to an individual skilled in the art (Greene, T. W.; Wuts, P. G. M. Protective Groups in Organic Synthesis; 3rd ed.; John Wiley & Sons: New York, 1999.) to avoid undesired interactions or reactions during the preparation of the individual building blocks and during library synthesis. Analogously, reactive elements may be masked by suitably selected protection groups. It is appreciated by one skilled in the art that by suitable protection, a functional entity may carry a wide range of substituents.

The Functional Entity Precursor may be masked Functional Entity that is incorporated into an encoded molecule. After incorporation, reactive elements of the Functional Entity may be revealed by un-masking allowing further synthetic operations. Finally, elements mediating recognition of host molecules may be un-masked.

The function of the carrier is to adjust the transferability of the functional entity, playing the role of a leaving group. Substituents on the carrier alter the leaving group efficiency. The stronger the electron withdrawing effect the easier the functional entity is cleaved from the remainder of the building block. However the cleavage can occur too fast which will result in unspecific transfer or hydrolysis. To adjust the transferability a skilled chemist can design suitable substitutions of the carrier by evaluation of initial attempts. The transferability may be adjusted in response to the chemical composition of the functional entity, to the nature of the complementing element, to the conditions under which the transfer and recognition is performed, ect.

According to a preferred embodiment of the invention the carrier is of the general formula:

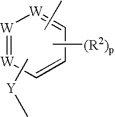

wherein W, Y, R², and p are as defined above. The transferability of the functional entity can be adjusted by suitable selection of the ring member. When the identity of W are fixed the transferability of the carrier may be adjusted by selecting type, position and amount of the ring substituents R². As an example, an unsubstituted benzene ring (W═CH for the entire ring structure) may be provided with an increased ability to transfer a functional entity by attaching a Cl in the ortho position. The ability to transfer functional entities may also be adjusted by proper selection of one, two or three nitrogen atoms in the ring structure. Finally, the identity and position of Y or alternatively the S—C-connecting group may have an influence of the transferability of the functional entity. Thus, attaching a carbonyl at the para position of the ring structure relative to the attachment point of the functional C—F-connecting group confers an increased ability to transfer the functional entity over a position in e.g. the meta position.

In a preferred aspect of the invention the carrier is

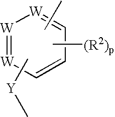

and attaches to the linker through Y and

W═CH

R²═—H, halogen, —NO₂, —CN, —C(Halogen)₃, —C(O)R³, —C(O)NHR³, C(O)NR³₂, —S(O)₂NHR³, —S(O)₂NR³₂, —S(O)₂R³, —N⁺R³₃, wherein halogen is selected from the group consisting of —Cl, —F, —Br, and —I, p is an integer of 0 to 3, and R³═H, C₁-C₆ alkyl, or aryl,

Y=absent, C₁-C₆ Alkylene, or carbonyl.

The spacer serves to distance the functional entity to be transferred from the bulky complementing element. Thus, when present, the identity of the spacer is not crucial for the function of the building block. It may be desired to have a spacer which can be cleaved by light. In this occasion, the spacer is provided with e.g. the group

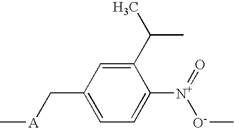

In the event an increased hydophilicity is desired the spacer may be provided with a polyethylene glycol part of the general formula:

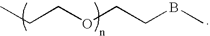

In a certain aspect of the invention the Spacer is a valence bond, C₁-C₆ alkylene-A-, C₁-C₆ alkenylene-A-, C₂-C₆ alkynylene-A-, or

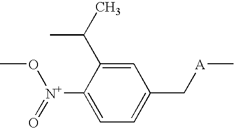

said spacer optionally being connected through A to a linker selected from

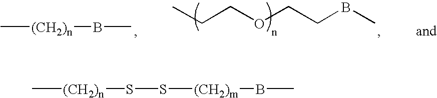

where A is —C(O)NR¹—, —NR¹—, —O—, —S—, or —C(O)—O—; B is —O, —S—, —NR¹— or —C(O)NR¹— and connects to S—C-connecting group; R¹ is selected independently from H, C₁-C₆ alkyl, C₃-C₇ cycloalkyl, C₁-C₆ alkylene-aryl, or aryl substituted with 0-5 halogen atoms selected from —F, —Cl, —Br and —I; and n and m independently are integers ranging from 1 to 10.

More preferred the Spacer is C₁-C₆ alkylene-A-, C₁-C₆ alkenylene-A-, C₂-C₆ alkynylene-A-, or

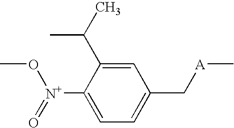

said spacer optionally being connected through A to a moiety selected from

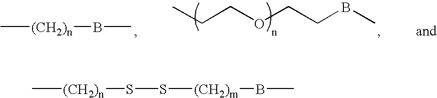

where A is —C(O)NR¹—, or —S—; B is —S—, —NR¹— or —C(O)NR¹— and connects to S—C-connecting group; R¹ is selected independently from H, C₁-C₆ alkyl, C₁-C₆ alkylene-aryl, or aryl; and n and m independently are integers ranging from 1 to 6.

In certain other aspects of the invention the Spacer is -A-, a group C₁-C₆ alkylene-A-, C₂-C₆ alkenylene-A-, or C₂-C₆ alkynylene-A-optionally substituted with 1 to 3 hydroxy groups, or

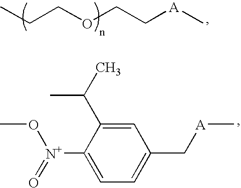

said spacer being connected through A to a linker selected from

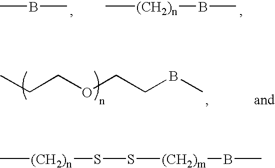

where A is a valence bond, —NR¹⁰—, —C(O)NR¹⁰—, —NR¹⁰—C(O)—, —O—, —S—, —C(O)—O— or —OP(═O)(O⁻)—O—; B is a valence bond, —O—, —S—, —NR¹⁰—, —C(O)— or —C(O)NR¹⁰— and connects to S—C-connecting group; R¹⁰ is selected independently from H, C₁-C₆ alkyl, C₃-C₇ cycloalkyl, aryl, C₁-C₆ alkylene-aryl, 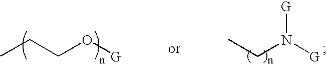
G is H or C₁-C₆ alkyl; and n and m independently are integers ranging from 1 to 10.

In a preferred aspect of the invention, the spacer is C₂-C₆ alkenylene-A, said spacer being connected through A to a moiety selected from

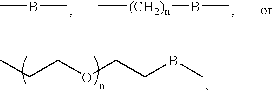

where A is a valence bond, —C(O)NR¹⁰—, —NR¹⁰—C(O)—, —S—, —C(O)—O— or —OP(═O)(O⁻)—O—; B is a valence bond, —S—, —NR¹⁰—, or —C(O)— and connects to S—C-connecting group; n and m independently are integers ranging from 1 to 10 and

R¹⁰ is selected independently from H, 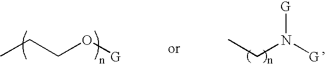
wherein G is H or C₁-C₆ alkyl; and the spacer is connected to the complementing element through a nucleobase.

Usually, the spacer connects to the 5 position of a pyrimidine or the 7 position of a purine or deaza-purine. However, other attachment point on the nucleobase may be contemplated.

In another preferred aspect the spacer connects to the back bone of the complementing element. In this case the spacer is -A-,

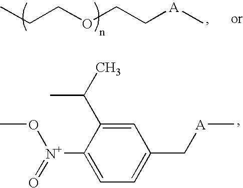

said spacer being connected through A to a moiety selected from

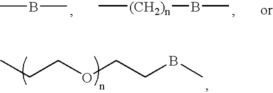

where A is a valence bond, —NR¹⁰—C(O)—, —O—, or —S—; B is a valence bond, —S—, —NR¹⁰—, or —C(O)— and connects to S—C-connecting group;

n and m independently are integers ranging from 1 to 10 and

R¹⁰ is selected independently from H, 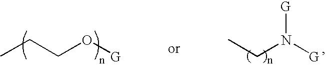
wherein G is H or C₁-C₆ alkyl; and the spacer is connected to the complementing element via a phosphorus group.

The phosphorus group is preferably a phosphate or a thiophosphate group attached to a 3′ or a 5′ end of a complementing element.

In a preferred embodiment, the complementing element serves the function of transferring genetic information e.g. by recognising a coding element. The recognition implies that the two parts are capable of interacting in order to assemble a complementing element—coding element complex. In the biotechnological field a variety of interacting molecular parts are known which can be used according to the invention. Examples include, but are not restricted to protein-protein interactions, protein-polysaccharide interactions, RNA-protein interactions, DNA-DNA interactions, DNA-RNA interactions, RNA-RNA interactions, biotin-streptavidin interactions, enzyme-ligand interactions, antibody-ligand interaction, protein-ligand interaction, ect.

The interaction between the complementing element and coding element may result in a strong or a week bonding. If a covalent bond is formed between the parties of the affinity pair the binding between the parts can be regarded as strong, whereas the establishment of hydrogen bondings, interactions between hydrophobic domains, and metal chelation in general results in weaker bonding. In general relatively weak bonding is preferred. In a preferred aspect of the invention, the complementing element is capable of reversible interacting with the coding element so as to provide for an attachment or detachment of the parts in accordance with the changing conditions of the media.

In a preferred aspect of the invention, the interaction is based on nucleotides, i.e. the complementing element is a nucleic acid. Preferably, the complementing element is a sequence of nucleotides and the coding element is a sequence of nucleotides capable of hybridising to the complementing element. The sequence of nucleotides carries a series of nucleobases on a backbone. The nucleobases may be any chemical entity able to be specifically recognized by a complementing entity. The nucleobases are usually selected from the natural nucleobases (adenine, guanine, uracil, thymine, and cytosine) but also the other nucleobases obeying the Watson-Crick hydrogen-bonding rules may be used, such as the synthetic nucleobases disclosed in U.S. Pat. No. 6,037,120. Examples of natural and non-natural nucleobases able to perform a specific pairing are shown in FIG. 2. The backbone of the sequence of nucleotides may be any backbone able to aggregate the nucleobases is a sequence. Examples of backbones are shown in FIG. 4. In some aspects of the invention the addition of non-specific nucleobases to the complementing element is advantageous, FIG. 3.

The coding element can be an oligonucleotide having nucleobases which complements and is specifically recognised by the complementing element, i.e. in the event the complementing element contains cytosine, the coding element part contains guanine and visa versa, and in the event the complementing element contains thymine or uracil the coding element contains adenine.

The complementing element may be a single nucleobase. In the generation of a library, this will allow for the incorporation of four different functional entities into the template-directed molecule. However, to obtain a higher diversity a complementing element preferably comprises at least two and more preferred at least three nucleotides. Theoretically, this will provide for 4² and 4³, respectively, different functional entities uniquely identified by the complementing element. The complementing element will usually not comprise more than 100 nucleotides. It is preferred to have complementing elements with a sequence of 3 to 30 nucleotides. The spacer part of the linker is attached to the carrier through a S—C-connecting group (short for Spacer-Carrier-connecting group). The S—C-connecting may have any chemical composition which provides for an attachment of the Spacer with the carrier. In certain aspect of the invention the S—C-connecting group is a valence bond, —NH—C(═O)—, —NH—C(═O)—C₁-C₆ alkylene-, —S—S—, —S—S—C₁-C₆ alkylene-, —C₁-C₆ alkylene-S—S—, —C(═O)—NH—(C₁-C₆ alkylene)-,

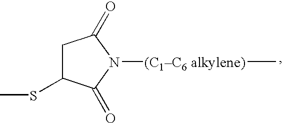

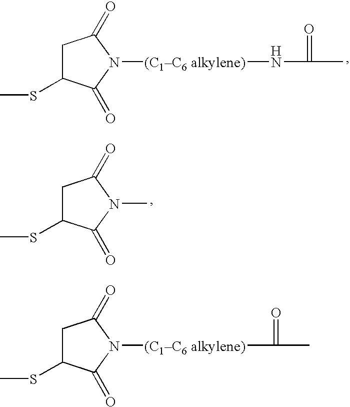

—NH—C(═O)-Arylene-C(R¹⁰)₂—NH—C(═O)—, —C(═O)—, —C(═O)—C₁-C₆ alkylene- or —C(═O)— Arylene-C(R¹⁰)₂—NR¹⁰—C(═O)—, where the right hand side of the formulae connects to the carrier.

In a preferred aspect the S—C-connecting group is —S—S—, —C₁-C₆ alkylene-S—S—, —C(═O)—NH—(C₁-C₆ alkylene)-, —C(═O)—, or —C(═O)-Arylene-C(R¹⁰)₂—NR¹⁰—C(═O)—, where the right hand side of the formulae connects to the carrier.

In a still more preferred aspect the S—C-connecting group is a valence bond, —NH—C(═O)—, —S—S—, or —C(═O)—NH—, where the right hand side of the formulae connects to the carrier.

The building blocks of the present invention can be used in a method for transferring a functional entity to a recipient reactive group, said method comprising the steps of providing one or more building blocks as described above and contacting the one or more building blocks with a corresponding encoding element associated with a recipient reactive group under conditions which allow for a recognition between the one or more complementing elements and the encoding elements, said contacting being performed prior to, simultaneously with, or subsequent to a transfer of the functional entity to the recipient reactive group.

The encoding element may comprise one, two, three or more codons, i.e. sequences that may be specifically recognised by a complementing element. Each of the codons may be separated by a suitable spacer group. Preferably, all or at least a majority of the codons of the template are arranged in sequence and each of the codons are separated from a neighbouring codon by a spacer group. Generally, it is preferred to have more than two codons on the template to allow for the synthesis of more complex encoded molecules. In a preferred aspect of the invention the number of codons of the encoding element is 2 to 100. Still more preferred are encoding elements comprising 3 to 10 codons. In another aspect, a codon comprises 1 to 50 nucleotides and the complementing element comprises a sequence of nucleotides complementary to one or more of the encoding sequences.

The recipient reactive group may be associated with the encoding element in any appropriate way. Thus, the reactive group may be associated covalently or non-covalently to the encoding element. In one embodiment the recipient reactive group is linked covalently to the encoding element through a suitable linker which may be separately cleavable to release the reaction product. In another embodiment, the reactive group is coupled to a complementing element, which is capable of recognising a sequence of nucleotides on the encoding element, whereby the recipient reactive group becomes attached to the encoding element by hybridisation. Also, the recipient reactive group may be part of a chemical scaffold, i.e. a chemical entity having one or more reactive groups available for receiving a functional entity from a building block.

The recipient reactive group may be any group able to cleave the C—F-connecting group to release the functional entity. Usually, the reactive group is nucleophilic, such as a hydroxyl, a thiol, an amine ect. A preferred recipient reactive group is an amine group. The nucleophile usually attacks the C—F-connecting group between Z and X═V or between the carrier and X═V, thereby causing the carrier group with an optional Z group to be the leaving group of the reaction and transferring the X(═V)—Functional entity precursor to the recipient. The chemical structure formed has, in the event the nucleophilic group is an amine attached to a scaffold, the general formula:
Scaffold-NH—X(═V)-Functional entity precursor

In which
X═—C—, —S—, —P—, —S(O)—, —P(O)—, and
V═O, S, NH, N—C₁-C₆ alkyl.

In a preferred aspect X is —C— and V is O.

The conditions which allow for transfer to occur are dependent upon the building block, notable the carrier and the C—F-connecting group, as well as the receiving reactive group. Below various examples of the conditions for a transfer to occur are depicted together with the reaction product formed.

A. Acylating Building Blocks—Principle

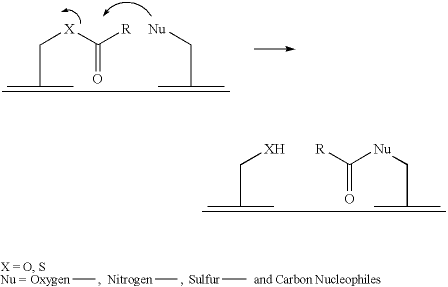

B. Amide Formation by Reaction of Amines with Activated Esters

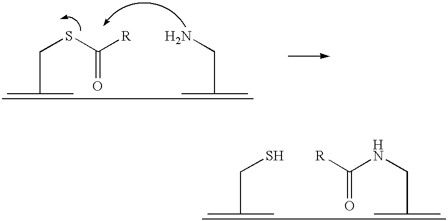

C. Pyrazolone Formation by Reaction of Hydrazines with β-Ketoesters

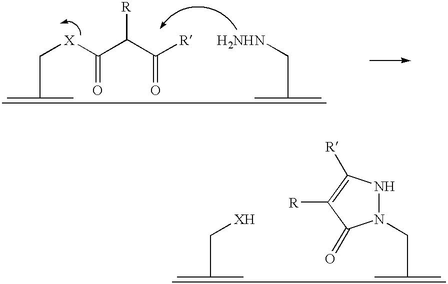

D. Isoxazolone Formation by Reaction of Hydroxylamines with β-Ketoesters

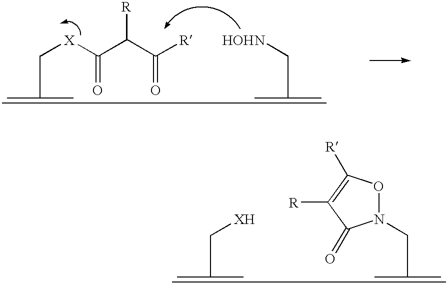

E. Pyrimidine Formation by Reaction of Thioureas with β-Ketoesters

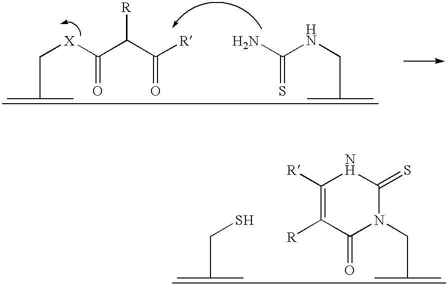

F. Pyrimidine Formation by Reaction of Ureas with Malonates

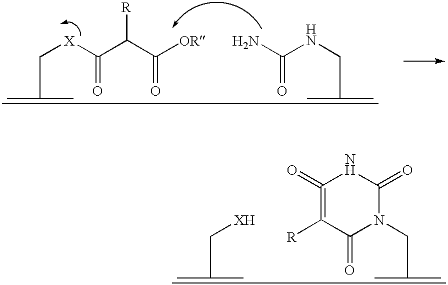

G. Coumarine or Quinolinon Formation by a Heck Reaction Followed by a Nucleophilic Substitution

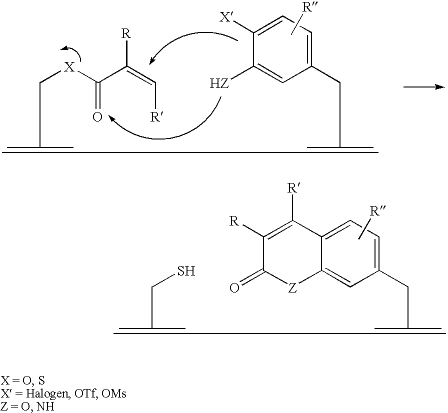

H. Phthalhydrazide Formation by Reaction of Hydrazines and Phthalimides

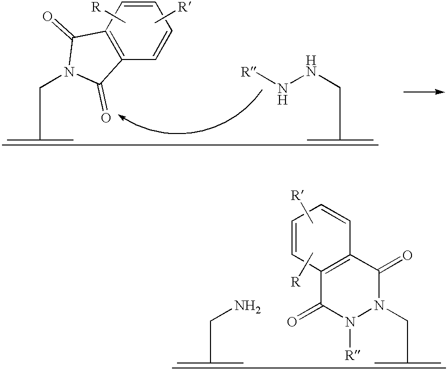

I. Diketopiperazine Formation by Reaction of Amino Acid Esters

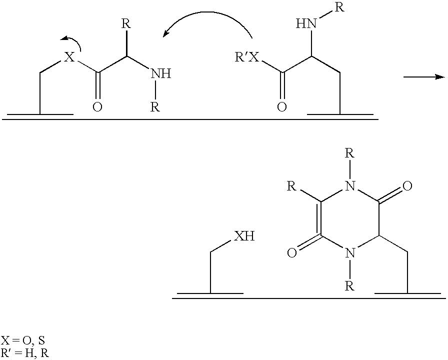

J. Hydantoin Formation by Reaction of Urea and α-Substituted Esters

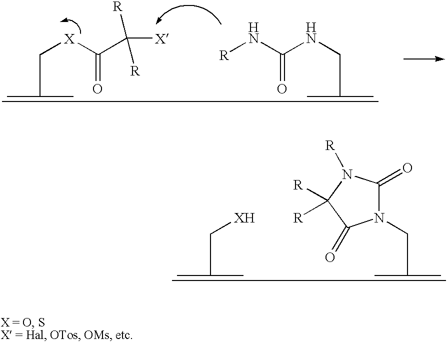

According to a preferred aspect of the invention the building blocks are used for the formation of a library of compounds. The complementing element of the building block is used to identify the functional entity. Due to the enhanced proximity between reactive groups when the complementing entity and the encoding element are contacted, the functional entity together with the identity programmed in the complementing element is transferred to the encoding element associated with recipient reactive group. Thus, it is preferred that the sequence of the complementing element is unique in the sense that the same sequence is not used for another functional entity. The unique identification of the functional entity enable the possibility of decoding the encoding element in order to determine the synthetic history of the molecule formed. In the event two or more functional entities have been transferred to a scaffold, not only the identity of the transferred functional entities can be determined. Also the sequence of reaction and the type of reaction involved can be determined by decoding the encoding element. Thus, according to a preferred embodiment of the invention, each different member of a library comprises a complementing element having a unique sequence of nucleotides, which identifies the functional entity.

### BRIEF DESCRIPTION OF THE DRAWINGS

FIG. 1 shows to setups for functional entity transfer.

FIG. 2 shows examples of specific base pairing.

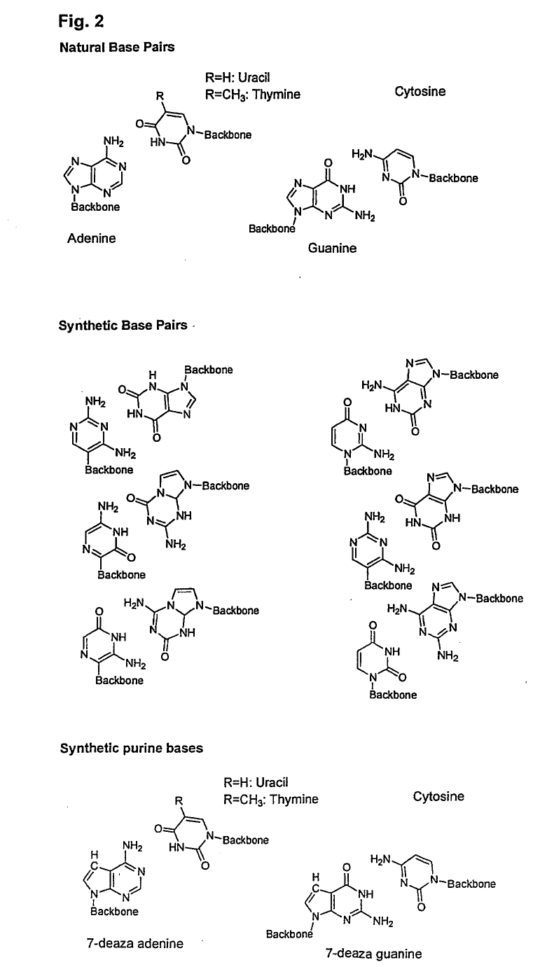

FIG. 3 shows examples of non-specific base-pairing

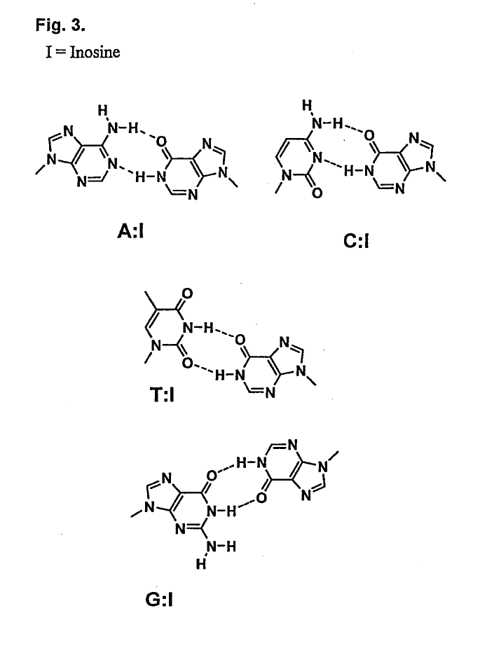

FIG. 4 shows examples of backbones.

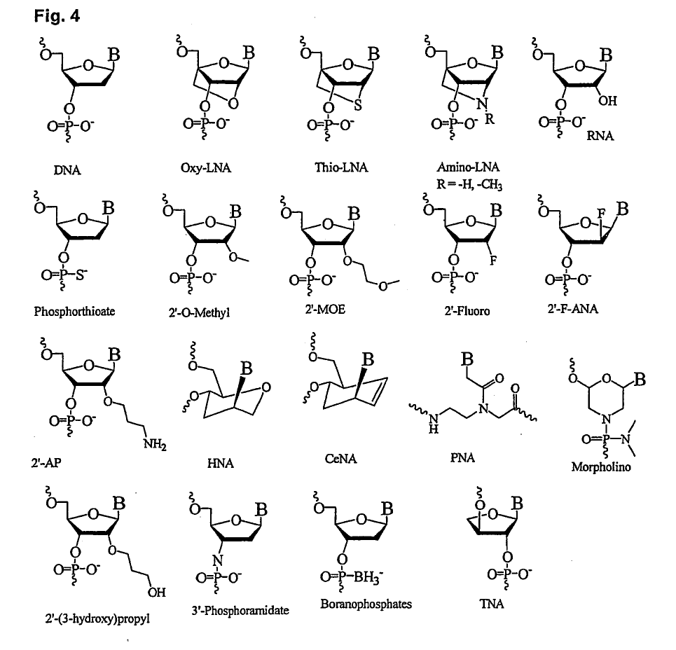

FIG. 5 shows a gel with the results of the experiments reported in example 22.

FIG. 6 shows three examples of building block according to the present invention.

### DETAILED DESCRIPTION OF THE INVENTION

A building block of the present invention is characterized by its ability to transfer its functional entity to a receiving chemical entity. This is done by forming a new covalent bond between the receiving chemical entity and cleaving the bond between the carrier moiety and the functional entity of the building block.

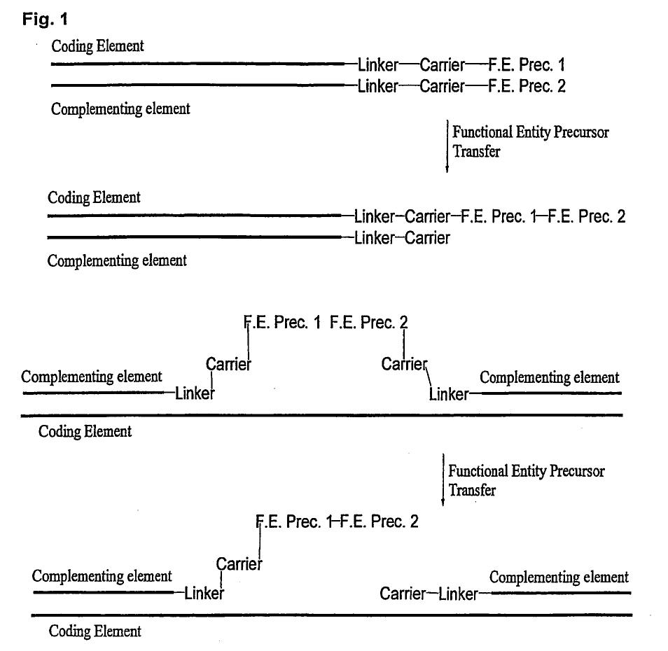

Two setups for generalized functional entity transfer from a building block are depicted in FIG. 1. In the first example, one complementing element of a building block recognizes a template carrying another functional entity, hence bringing the functional entities in close proximity. This results in a reaction between functional entity precursor 1 and 2 forming a covalent bond between these concurrent with the cleavage of the bond between functional entity precursor 2 and its linker. In the second example, a template brings together two building blocks resulting in functional entity transfer from one building block to the other.

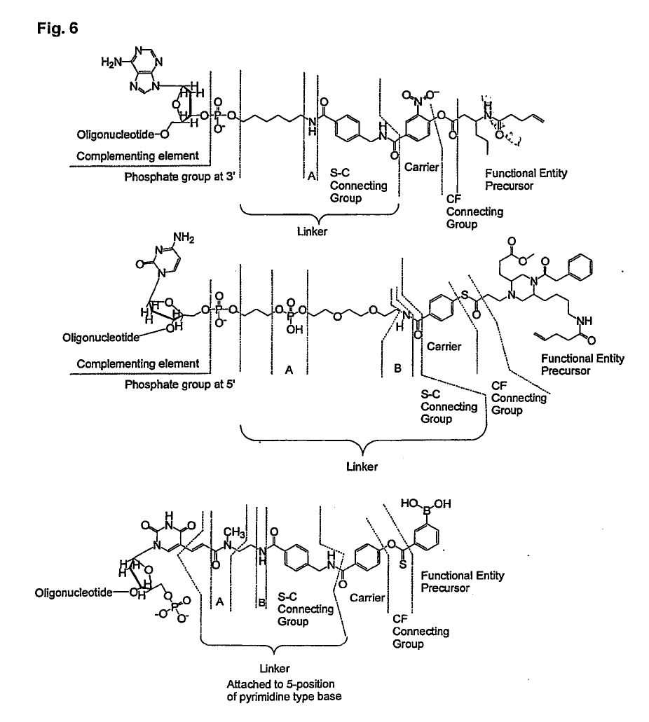

FIG. 6 discloses three examples of building blocks. For illustrative purposes the individual features used in the claims are indicated. In the upper compound the spacer part of the linker connects to a 3′-phosphate group of an oligonucleotide. The first part of the linker, i.e. the spacer, is an aliphatic chain ending in a nitrogen atom. The nitrogen atom bridges to the S—C-connecting group, which is an N-acylated arylmethyleamine. The carrier attached to the left hand side carbonyl group of the S—C-connecting group is a nitrophenyl group. In the para position of the nitrophenyl group, the C-F-conneting group is attached. When the building block is presented to a nucleophilic group, the functional entity precursor and the carbonyl group of the C—F-connecting group is transferred. In the event the nucleophilic group is an amine, the bond formed is an amide bond.

The middle compound of FIG. 6 discloses a linker attached to the 5′ position of an oligonucleotide. The linker is attached through a 5′ phosphate group and extends into a short 3 member aliphatic chain to another phosphate group which is connected to a linker terminal nitrogen group via a PEG part. The linker nitrogen group is connected to the carrier via a carbonyl group. The carrier is of the thiophenyl type as the sulphur of the C—F-connecting group connects to the ring structure. When the building block is presented to a nucleophilic group, such as an amine, the functional entity precursor together with the carbonyl group of the C—F-connecting group is transferred to said recipient group forming an amide bond when the nucleophile is an amine.

The lower compound shown on FIG. 6 illustrates an example of the linker being connected to the nucleobase of the oligonucleotide complementing element. More specifically, the linker connects to the 5 position of a pyrimidine. The linker extents through an α-β unsaturated N-methylated amide to the S—C-connecting group, which is a 4-amino methyl benzoic acid derivative. The carrier is of the phenol type and the functional entity precursor together with the thiocarbonyl group of the C—F-connecting group may be transferred to a recipient reactive group forming an amide in the event the recipient reactive group is an amine.

In a library synthesis, several building blocks are mixed in a reaction vessel and the added templates ensure that the building blocks—consequently the functional entities—are combined in the desired manner. As several building blocks are employed at the same time, the use of in situ generated building blocks is disfavoured for practical reasons.

Building blocks for library synthesis should posses the necessary reactivity to enable the transfer of the functional entity but should also be stable enough to endure storage and the conditions applied during library synthesis. Hence fine tuning of the reactivity for a particular building block is vital. The reactivity of a building block depends partly on the characteristics of the functional entity and the characteristics of the carrier. E.g. a highly reactive functional entity attached to a highly reactive carrier would form a building block that may be susceptible to hydrolysis during the library synthesis thus preventing successful transfer of one functional entity to another. Further, if transfer of a functional entity precursor is faster than coding element—complementing element recognition unspecific reactions may result. Therefore, the present invention particularly relates to practically useful library building blocks capable of acting as acylating agents, thioacetylating agents or amidinoylating agents with a balanced reactivity. Such building blocks may be assembled by several different pathways as described below.

Formation of an Amide Bond Between a Carboxylic Acid of the Carrier and an Amine Group of a Spacer

The Carrier-Functional Entity Precursor ensemble may be bound to the Spacer by several different reactions as illustrated below.

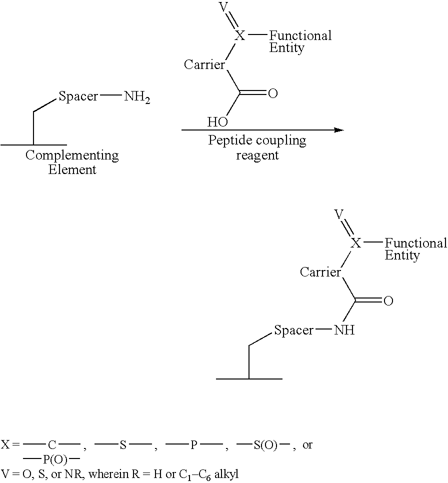

Examples of Carrier-Functional Entity Precursor reagents:

| | |
|---|---|
| 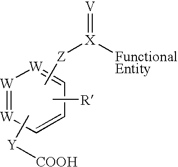 | Z = O, S X = —C—,—S—, —P—, —S(O)—, —P(O)—V = O, S, NR, R = H, C₁-C₆ alkyl W = CH or N, chosen independently R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, |
| # —P(O)₂—R″, —P(O)—R″, —S(O)—R″, P(O)—OR″, —S(O)—OR″, —N⁺R″₃, R″ = H, C₁-C₆ alkyl, C₁-C₆ alkenyl, C₁-C₆ alkynyl or aryl, chosen independently Y = absent, C₁-C₆ Alkylene, C₁-C₆ Alkenylene, C₁-C₆ Alkynylene, Arylene, Heteroarylene, Carbonyl, —SO₂CH₂— | |
| 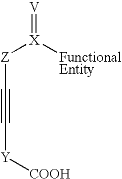 | Z = O, S X = —C—, —S—, —P—, —S(O)—, —P(O)—V = O, S, NR, R═H, C₁-C₆ alkyl Y = absent, C₁-C₆ Alkyl, C₁-C₆ Alkenyl, C₁-C₆ Alkynyl, Aryl, Heteroaryl, Carbonyl, —SO₂CH₂— |
| 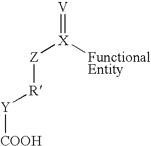 | Z = S R′ = —CH₂—X = —C—, —S—, —P—, —S(O)—, —P(O)—V = O, S, NR, R—H, C₁-C₆ alkyl Y = nothing, C₁-C₆ Alkylene, C₁-C₆ Alkenylene, C₁-C₆ Alkynylene, Arylene, Heteroarylene, Carbonyl, —SO₂CH₂— |
| 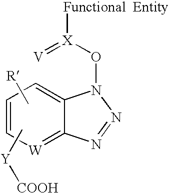 | W = CH or N X = —C—, —S—, —P—, —S(O)—, —P(O)—V = O, S, NR, R═H, C₁-C₆ alkyl R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, |
| # —P(O)₂—R″, —P(O)—R″, —S(O)—R″, P(O)—OR″, —S(O)—OR″, —N⁺R″₃, R″ = alkyl, alkenyl, alkynyl, aryl. Y = nothing, C₁-C₆ Alkyl, C₁-C₆ Alkenyl, C₁-C₆ Alkynyl, Aryl, Heteroaryl, Carbonyl, —SO₂CH₂— | |
| 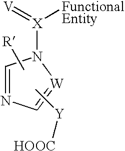 | W = CH or N X = —C—, —S—, —P—, —S(O)—, —P(O)—V = O, S, NR, R═H, C₁-C₆ alkyl R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, |
| # —P(O)₂—R″, —P(O)—R″, —S(O)—R″, P(O)—OR″, —S(O)—OR″, —N⁺R″₃, R″ = H, C₁-C₆ alkyl, C₁-C₆ alkenyl, C₁-C₆ alkynyl or aryl, chosen independently Y = absent, C₁-C₆ Alkylene, C₁-C₆ Alkenylene, C₁-C₆ Alkynylene, | |
| # Arylene, Heteroarylene, Carbonyl, —SO₂CH₂— | |
| 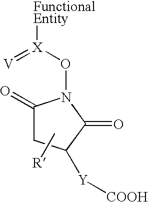 | X = —C—, —S—, —P—, —S(O)—, —P(O)—V = O, S, NR, R═H, C₁-C₆ alkyl R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P(O)—R″, |
| # —S(O)—R″, P(O)—OR″, —S(O)—OR″, —N⁺R″₃, R″ = H, C₁-C₆ alkyl, C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |
| 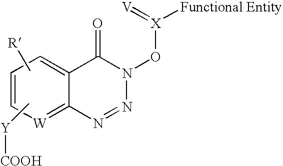 | W = CH or N X = —C—, —S—, —P—, —S(O)—, —P(O)—V = O, S, NR, R═H, C₁-C₆ alkyl R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, |
| # —P(O)₂—R″, —P(O)—R″, —S(O)—R″, P(O)—OR″, —S(O)—OR″, —N⁺R″₃, R″ = alkyl, alkenyl, alkynyl, aryl. Y = nothing, C₁-C₆ Alkyl, C₁-C₆ Alkenyl, C₁-C₆ Alkynyl, Aryl, Heteroaryl, Carbonyl, —SO₂CH₂— | |
| 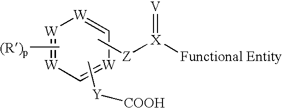 | Z = O, S X = —C—, —S—, —P—, —S(O)—, —P(O)—V = O, S, NR, R═H, C₁-C₆ alkyl W = CH or N, chosen independently R′ = —H, —Halogen, —NO₂, —CN, —C(Halogen)₂, —C(O)R″, —C(O)NHR″, C(O)NR″₂, —NC(O)R″, —S(O)₂NHR″, —S(O)₂NR″₂, |
| # —S(O)₂R″, —P(O)₂—R″, —P(O)—R″, —S(O)—R″, P(O)—OR″, —S(O)—OR″, —N⁺R″₃chosen independently p = 0, 1, 2, 3 or 4 Y = absent, C₁-C₆ Alkylene, C₁-C₆ Alkenylene, C₁-C₆Alkynylene, Arylene, Heteroarylene, Carbonyl, —SO₂CH₂— | |

Stepwise Loading of the Carrier and the Functional Entity

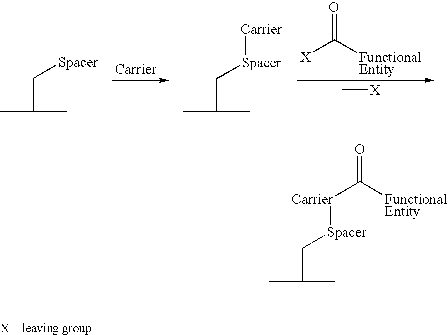

Sequential loading of the carrier and the functional entity allows other types of chemistries to be used.

Carrier Introduced Via Amide Bond Formation

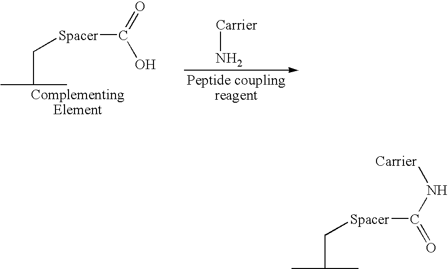

Examples of Carrier Reactants:

| | |
|---|---|
| 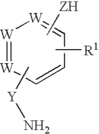 | Z = O, S W = CH or N, independently chosen R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P(O)—R″, —S(O)—R″, P(O)—OR″, —S(O)—OR″, |
| # —N⁺R″₃, R″ = H, C₁-C₆ alkyl, C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |
| 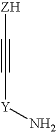 | Z = O, S Y = nothing, C₁-C₆ Alkyl, C₁-C₆ Alkenyl, C₁-C₆ Alkynyl, Aryl, Heteroaryl, Carbonyl, —SO₂CH₂— |
| 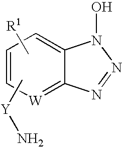 | W = CH or N R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P(O)—R″, —S(O)—R″, P(O)—OR″, —S(O)—OR″, —N⁺R″₃, R″ = H, |
| # C₁-c₆ alkyl, C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |
| 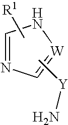 | W = CH or N R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P(O)—R″, —S(O)—R″, P(O)—OR″, —S(O)—OR″, —N⁺R″₃, R″ = H, |
| # C₁-C₆ alkyl, C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |
| 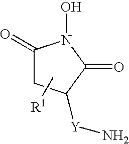 | R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P(O)—R″, —S(O)—R″, P(O)—OR″, —S(O)—OR″, —N⁺R″₃, R″ = H, C₁-C₆ alkyl, |
| # C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |

Carrier Introduced Via S—S Bond Formation

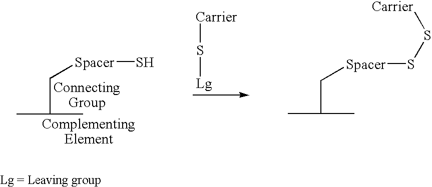

Examples of Carrier Reactants:

| | |
|---|---|
| 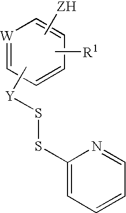 | Z = O W = CH or N R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P(O)—R″, —S(O)—R″, P(O)—OR″, —S(O)—OR″, —N⁺R″₃, R″ = H, |
| # C₁-C₆ alkyl, C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |
| 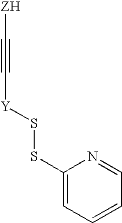 | Z = O Y = nothing, C₁-C₆ Alkyl, C₁-C₆ Alkenyl, C₁-C₆ Alkynyl, Aryl, Heteroaryl, Carbonyl, —SO₂CH₂— |
| 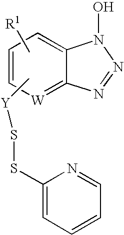 | W = CH or N R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P)O)—R″, —S(O)—R″, P(O)—OR″, —S(O)—OR″, —N⁺R″₃, R″ = H, |
| # C₁-C₆ alkyl, C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |
| 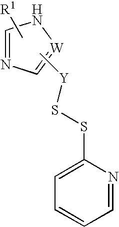 | W = CH or N R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P)O)—R″, —S(O)—R″, P(O)—OR″, —S(O)—OR″, —N⁺R″₃, R″ = H, |
| # C₁-C₆ alkyl, C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |
| 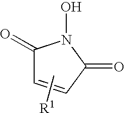 | R′ = —H, —Halogen, —NO₂, —CN, —C(Halogen)₃, —C(O)R″, —C(O)NHR″, C(O)NR″₂, —NC(O)R″, —S(O)₂NHR″, —S(O)₂NR″₂, —S(O)₂R″, —P(O)₂—R″, —P(O)—R″, —S(O)—R″, P(O)—OR″, —S(O)—OR″, —N⁺R″₃, R″ = H, C₁-C₆ alkyl, |
| # C₁-C₆ alkenyl, C₁-C₆ alkynyl or aryl, chosen independently Y = nothing, C₁-C₆ Alkylene, C₁-C₆ Alkenylene, C₁-C₆Alkynylene, Arylene, Heteroarylene, Carbonyl, —SO₂CH₂— | |

Functional Entity Introduced as a Thioacid

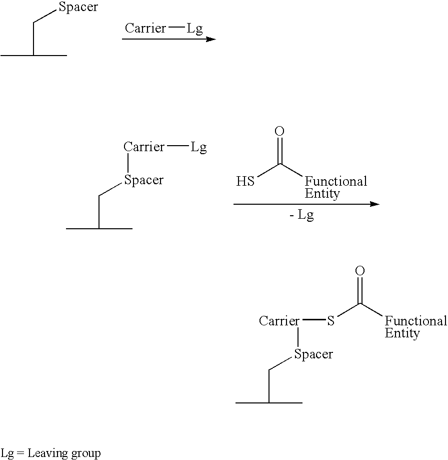

Examples of Carrier Reactants:

| | |
|---|---|
| 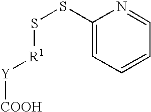 | R′ = —CH₂—Y = nothing, C₁-C₆ Alkylene, C₁-C₆Alkenylene, C₁-C₆ Alkynylene, Arylene, Heteroarylene, Carbonyl, —SO₂CH₂— |

As discussed above the C—F-connecting group may be selected from a large group of compounds of the general formula -Z-(X═V)— or —X═V)—. In certain aspects of the invention X═C, S, P, S(═O), or P(═O), in another preferred embodiment X═C, S, or S(═O), and in still another preferred embodiment X═C. In certain aspects of the invention V═O, S, NR¹⁰ or NOR¹⁰, in another preferred embodiment V═O or NR¹⁰, and in still another preferred embodiment V═O. In a certain aspect of the invention Z=O, or S, in another preferred embodiment, Z=O, and in still another preferred embodiment, Z=S.

Wherein R¹⁰ is H, alkyl, alkenyl, alkynyl, alkadienyl, cycloalkyl, cycloheteroalkyl, aryl or heteroaryl, optionally substituted with one or more substituents selected from the group consisting of SnR¹¹R¹², R¹³, Sn(OR¹¹)R¹²R¹³, Sn(OR¹¹)(OR¹²)R¹³, BR¹¹R¹², B(OR¹¹)R¹², B(OR¹¹)(OR¹²), halogen, CN, CNO, C(halogen)₃, OR¹¹, OC(═O)R¹¹, OC(═O)OR¹¹, OC(═O)NR¹¹R¹², SR¹¹, S(═O)R¹¹, S(═O)₂R¹¹, S(═O)₂NR¹¹R¹², NO₂, N₃, NR¹¹R¹², N⁺R¹¹R¹²R¹³, NR¹¹OR¹², NR¹¹NR¹²R¹³, NR¹¹C(═O)R¹², NR¹¹C(═O)OR¹², NR¹¹C(═O)NR¹²R¹³, NC, P(═O)(O¹¹)OR¹², P⁺R¹¹R¹²R¹³, C(═O)R¹¹, C(═NR¹¹)R¹², C(═NOR¹¹)R¹², C(═NNR¹¹R¹²), C(═O)OR¹¹, C(═O)NR¹¹R¹², C(═O)NR¹¹OR¹², C(═O)NR¹¹NR¹²R¹³, C(═NR¹¹)NR¹²R¹³, C(═NOR¹¹)NR¹²R¹³ or R¹⁴,

wherein,

R¹¹, R¹² and R¹³ independently is H, alkyl, alkenyl, alkynyl, alkadienyl, cycloalkyl, cycloheteroalkyl, aryl or heteroaryl, optionally substituted with one or more substituents selected from the group consisting of halogen, CN, CNO, C(halogen)₃, OR¹⁴, OC(═O)R¹⁴, OC(═O)OR¹⁴, OC(═O)NR¹⁴R¹⁵, SR¹⁴, S(═O)R¹⁴, S(═O)₂R¹⁴, S(═O)₂NR¹⁴R¹⁵, NO₂, N₃, NR¹⁴R¹⁵, N⁺R¹⁴R¹⁵R¹⁶, NR¹¹OR¹², NR¹¹R¹²R¹³, NR¹⁴C(═O)R¹⁵, NR¹⁴C(═O)OR¹⁵, NR¹⁴C(═O)NR¹⁵R₁₆, NC, P(═O)(OR¹⁴)OR¹⁵, P⁺R¹¹R¹²R¹³, C(═O)R¹⁴, C(═NR¹⁴)R¹⁵, C(═NOR¹⁴)R¹⁵, C(═NNR¹⁴R¹⁵), C(═O)OR¹⁴, C(═O)NR¹⁴R¹⁵, C(═O)NR¹⁴OR¹⁵C(═NR¹¹)NR¹²R¹³, C(═NOR¹¹)NR¹²R¹³ or C(═O)NR¹⁴NR¹⁵R¹⁶, wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

wherein,

R¹⁴, R¹⁵ and R¹⁶ independently is H, alkyl, alkenyl, alkynyl, alkadienyl, cycloalkyl, cycloheteroalkyl, aryl or heteroaryl and wherein R¹⁴ and R¹⁵ may together form a 3-8 membered heterocyclic ring or R¹⁴ and R¹⁵ may together form a 3-8 membered heterocyclic ring or R¹⁵ and R¹⁶ may together form a 3-8 membered heterocyclic ring,

in a further preferred embodiment,

R¹⁰ is H, C₁-C₆ alkyl, C₂-C₆ alkenyl, C₂-C₆ alkynyl, C₄-C₆ alkadienyl, C₃-C₇ cycloalkyl, C₃-C₇ cycloheteroalkyl, aryl or heteroaryl, optionally substituted with one or more substituents selected from the group consisting of SnR¹¹R¹², R¹³, Sn(OR¹¹)R¹²R¹³, Sn(OR¹¹)(OR¹²)R¹³, BR¹¹R¹², B(OR¹¹)R¹², B(OR¹¹)(OR¹²), halogen, CN, CNO, C(halogen)₃, OR¹¹, OC(═O)R¹¹, OC(═O)OR¹¹, OC(═O)NR¹¹R¹², SR¹¹, S(═O)R¹¹, S(═O)₂R¹¹, S(═O)₂NR¹¹R¹², NO₂, N₃, NR¹¹R¹², N⁺R¹¹R¹²R¹³, NR¹¹OR¹², NR¹¹NR¹²R¹³, NR¹¹C(═O)R¹², NR¹¹C(═O)OR¹², NR¹¹C(═O)NR¹²R¹³, NC, P(═O)(OR¹¹)OR¹², P⁺R¹¹R¹²R¹³, C(═O)R¹¹, C(═NR¹¹)R¹², C(═NOR¹¹)R¹², C(═NNR¹¹R¹²), C(═O)OR¹¹, C(═O)NR¹¹R¹², C(═O)NR¹¹OR¹², C(═O)NR¹¹NR¹²R¹³, C(═NR¹¹)NR¹²R¹³, C(═NOR¹¹)NR¹²R¹³ or R¹⁴,

wherein,

R¹¹, R¹², R¹³ and R¹⁴ independently is H, C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in another preferred embodiment,

R¹⁰ is H, C₁-C₆ alkyl, C₃-C₇ cycloalkyl, C₃-C₇ cycloheteroalkyl, aryl or heteroaryl, optionally substituted with one or more substituents selected from the group consisting of halogen, CN, C(halogen)₃, OR¹¹, OC(═O)R¹¹, OC(═O)OR¹¹, OC(═O)NR¹¹R¹², SR¹¹, S(═O)R¹¹, S(═O)₂R¹¹, S(═O)₂NR¹¹R¹², NO₂, NR¹¹R¹², NR¹¹NR¹²R¹³, NR¹¹C(═O)R¹², NR¹¹C(═O)OR¹², NR¹¹C(═O)NR¹²R¹³, P(═O)(OR¹¹)OR¹², C(═O)R¹¹, C(═NR¹¹)R¹², C(═NOR¹¹)R¹², C(═NNR¹¹R¹²), C(═O)OR¹¹, C(═O)NR¹¹R¹², C(═O)NR¹¹OR¹², C(═O)NR¹¹NR¹²R¹³, C(═NR¹¹)NR¹²R¹³, C(═NOR¹¹)NR¹²R¹³ or R¹⁴,

wherein,

R¹¹, R¹², R¹³ and R¹⁴ independently is H, C₁-C₆ alkyl, C₃-C₇ cycloalkyl, C₃-C₇ cycloheteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, C₁-C₆ alkyl, C₃-C₇ cycloalkyl, C₃-C₇ cycloheteroalkyl, aryl or heteroaryl, optionally substituted with one or more substituents selected from the group consisting of F, Cl, CN, CF₃, OR¹¹, OC(═O)R¹¹, OC(═O)OR¹¹, OC(═O)NR¹¹R¹², SR¹¹, S(═O)R¹¹, S(═O)₂R¹¹, S(═O)₂NR¹¹R¹², NO₂, NR¹¹R¹², NR¹¹OR¹², NR¹¹NR¹²R¹³, NR¹¹C(═O)R¹², NR¹¹C(═O)OR¹², NR¹¹C(═O)NR¹²R¹³, P(═O)(OR¹¹)OR¹², C(═O)R¹¹, C(═NR¹¹)R¹², C(═NOR¹¹)R¹², C(═NNR¹¹R¹²), C(═O)OR¹¹, C(═O)NR¹¹R¹², C(═O)NR¹¹OR¹², C(═O)NR¹¹NR¹²R¹³, C(═NR¹¹)NR¹²R¹³, C(═NOR¹¹)NR¹²R¹³ or R¹⁴

wherein,

R¹¹, R¹², R¹³ and R¹⁴ independently is H, C₁-C₈ alkyl, C₃-C₇ cycloalkyl, C₃-C₇ cycloheteroalkyl, aryl or heteroaryl and wherein R¹¹ and R¹² may together form a 3-8 membered heterocyclic ring or R¹² and R¹³ may together form a 3-8 membered heterocyclic ring or R¹² and R¹³ may together form a 3-8 membered heterocyclic ring,

in still another preferred embodiment,

R¹⁰ is H, C₁-C₆ alkyl, C₃-C₇ cycloalkyl, C₃-C₇ cycloheteroalkyl, aryl or heteroaryl, optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, C₁-C₆ alkyl, C₃-C₇ cycloalkyl, C₃-C₇ cyclo-heteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, methyl, ethyl, propyl, butyl, cyclopropyl, cyclobutyl, cyclopentyl, cyclohexyl, aziridinyl, azetidinyl, pyrrolidinyl, piperidinyl, morpholinyl, phenyl, naphtyl, thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, C₁-C₆ alkyl, C₃-C₇ cycloalkyl, C₃-C₇ cyclo-heteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, C₁-C₆ alkyl, C₃-C₇ cycloalkyl, C₃-C₇ cyclo-heteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, aziridinyl, azetidinyl, pyrrolidinyl, piperidinyl or morpholinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, C₁-C₆ alkyl, C₃-C₇ cycloalkyl, C₃-C₇ cyclo-heteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, phenyl, naphtyl, thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, C₁-C₆ alkyl, C₃-C₇ cycloalkyl, C₃-C₇ cyclo-heteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, phenyl or naphtyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, C₁-C₆ alkyl, C₃-C₇ cycloalkyl, C₃-C₇ cyclo-heteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, C₁-C₆ alkyl, C₃-C₇ cycloalkyl, C₃-C₇ cyclo-heteroalkyl, aryl or heteroaryl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, aziridinyl, azetidinyl, pyrrolidinyl, piperidinyl or morpholinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹¹, NR¹¹C(═O)OR¹², NR¹¹C(═O)NR¹²R¹³, C(═O)R¹¹, C(═NOR¹¹)R¹², C(═O)O^{R}¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, phenyl, naphtyl, thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, phenyl or naphtyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R₁₄,

wherein,

R¹¹, R¹², R¹³ and R¹⁴ independently is H, methyl, ethyl, propyl or butyl and wherein R¹¹ and R¹² may together form a 3-8 membered heterocyclic ring or R¹² and R¹³ may together form a 3-8 membered heterocyclic ring or R¹² and R¹³ may together form a 3-8 membered heterocyclic ring,

in still another preferred embodiment,

R¹⁰ is H, aziridinyl, azetidinyl, pyrrolidinyl, piperidinyl or morpholinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl or butyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, phenyl, naphtyl, thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl or butyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, phenyl or naphtyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl or butyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is H, thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, methyl, ethyl, propyl or butyl and wherein R¹¹ and R¹² may together form a 3-8 membered heterocyclic ring or R¹¹ and R¹³ may together form a 3-8 membered heterocyclic ring or R¹² and R¹³ may together form a 3-8 membered heterocyclic ring,

in still another preferred embodiment,

R¹⁰ is methyl, ethyl, propyl, butyl, cyclopropyl, cyclobutyl, cyclopentyl or cyclohexyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, cyclopropyl, cyclobutyl, cyclopentyl or cyclohexyl,

in still another preferred embodiment,

R¹⁰ is aziridinyl, azetidinyl, pyrrolidinyl, piperidinyl or morpholinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R₁₃, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, cyclopropyl, cyclobutyl, cyclopentyl or cyclohexyl,

in still another preferred embodiment,

R¹⁰ is phenyl, naphtyl, thienyl, furyl, pyridyl, quinolinyl or isoquinolinyl optionally substituted with one or more substituents selected from the group consisting of F. Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R₁₂, C(═O)OR¹¹, C(═b)NR¹¹R¹², C(═O)NR¹¹OR¹² or R¹⁴,

wherein,

R¹¹, R¹², R¹³ and R¹⁴ independently is H, cyclopropyl, cyclobutyl, cyclopentyl or cyclohexyl,

in still another preferred embodiment,

R¹⁰ is phenyl or naphtyl optionally substituted with one or more substituents selected from the group consisting of F, Cl, CN, CF₃, OR¹¹, S(═O)R¹¹, S(═O)₂R¹¹, S(═O)₂NR¹¹R₁₂, NO₂, NR¹¹R¹², NR¹¹C(═O)R², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, cyclopropyl, cyclobutyl, cyclopentyl or cyclohexyl,

in still another preferred embodiment,

R¹⁰ is thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, cyclopropyl, cyclobutyl, cyclopentyl or cyclohexyl,

in still another preferred embodiment,

R¹⁰ is methyl, ethyl, propyl, butyl, cyclopropyl, cyclobutyl, cyclopentyl or cyclohexyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, phenyl, naphthyl, thienyl, furyl, pyridinyl, quinolinyl or isoquinolinyl,

in still another preferred embodiment,

R¹⁰ is aziridinyl, azetidinyl, pyrrolidinyl, piperidinyl or morpholinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, phenyl, naphthyl, thienyl, furyl, pyridinyl, quinolinyl or isoquinolinyl,

in still another preferred embodiment,

R¹⁰ is phenyl, naphtyl, thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, phenyl, naphthyl, thienyl, furyl, pyridinyl, quinolinyl or isoquinolinyl,

in still another preferred embodiment,

R¹⁰ is phenyl or naphtyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR¹², NR¹¹C(═O)NR¹²R¹³, C(═O)R¹¹, C(═NOR¹¹)R¹², C(═O)OR¹¹, C(═O)NR¹¹R¹², C(═O)NR¹¹OR¹² or R¹⁴,

wherein,

R¹¹, R¹², R¹³ and R¹⁴ independently is H, phenyl, naphthyl, thienyl, furyl, pyridinyl, quinolinyl or isoquinolinyl,

in still another preferred embodiment,

R¹⁰ is thienyl, furyl, pyridyl, quinolinyl or isoquinolinyl optionally substituted with one or more substituents selected from the group consisting of F, Cl, CN, CF₃, OR¹¹, S(═O)R¹¹, S(═O)₂R¹¹, S(═O)₂NR¹¹R¹², NO₂, NR¹¹R¹², NR¹¹C(═O)R¹², NR¹¹C(═O)OR², NR¹¹C(═O)NR¹²R¹³, C(═O)R¹¹, C(═NOR¹¹)R¹², C(═O)O^{R}¹¹, C(═O)NR¹¹R¹², C(═O)NR¹¹OR¹² or R¹⁴,

wherein,

R¹¹, R¹², R¹³ and R¹⁴ independently is H, phenyl, naphthyl, thienyl, furyl, pyridinyl, quinolinyl or isoquinolinyl,

in still another preferred embodiment,

R¹⁰ is H, C₁-C₆ alkyl, C₃-C₇ cycloalkyl, C₃-C₇ cycloheteroalkyl, aryl or heteroaryl

in still another preferred embodiment,

R¹⁰ is H,

in still another preferred embodiment,

R¹⁰ is C₁-C₆ alkyl, C₃-C₇ cycloalkyl or C₃-C₇ cycloheteroalkyl,

in still another preferred embodiment,

R¹⁰ is methyl, ethyl, propyl or butyl

in still another preferred embodiment

R¹⁰ is cyclopropyl, cyclobutyl, cyclopentyl or cyclohexyl

in still another preferred embodiment

R¹⁰ is aziridinyl, pyrrolidinyl, piperidinyl or morpholinyl

in still another preferred embodiment,

R¹⁰ is aryl or heteroaryl

in still another preferred embodiment,

R¹⁰ is phenyl or naphthyl

in still another preferred embodiment,

R¹⁰ is thienyl, furyl, pyridyl, quinolinyl or isoquinolyl.

The Functional entity precursor may be selected from any transferable chemical group capable of forming a connection to the C—F-connecting group. In certain aspects of the invention the functional entity precursor is represented by the formula Z²R¹⁷

wherein Z is absent, O, S or NR²⁴. In certain embodiment Z is absent. In a another embodiment Z is O. In still another embodiment Z is S, and in still a further embodiment Z is NR²⁴.

R¹⁷ and R²⁴ independently is H, alkyl, alkenyl, alkynyl, alkadienyl, cycloalkyl, cycloheteroalkyl, aryl or heteroaryl, optionally substituted with one or more substituents selected from the group consisting of SnR¹⁸R¹⁹,R²⁰, Sn(OR¹⁸)R¹⁹R²⁰, Sn(OR¹⁸)(OR¹⁹)R²⁰, BR¹⁸R¹⁹, B(OR¹⁸)R¹⁹, B(OR¹⁸)(OR¹⁹), halogen, CN, CNO, C(halogen)₃, OR¹⁸, OC(═O)R¹⁸, OC(═O)OR¹⁸, OC(═O)NR¹⁸R¹⁹, SR¹⁸, S(═O)R¹⁸, S(═O)₂R¹⁸, S(═O)₂NR¹⁸R¹⁹, NO₂, N₃, NR¹⁸R¹⁹, N⁺R¹⁸R¹⁹R, NR²⁰, NR¹⁸OR¹⁹, NR¹⁸NR¹⁹R²⁰, NR¹⁸C(═O)R¹⁹, NR¹⁸C(═O)OR¹⁹ NR¹⁸C(═O)NR¹⁹R²⁰, NC, P(═O)(OR¹⁸)OR¹⁹, P⁺R¹⁸R¹⁹R²⁰, C(═O)R¹⁸, C(═NR¹⁸)R¹⁹, C(═NOR¹⁸)R¹⁹, C(═NNR¹⁸R¹⁹), C(═O)OR¹⁸, C(═O)NR¹⁸R¹⁹, C(═O)NR¹⁸OR¹⁹, C(═O)NR¹⁸NR¹⁹R²⁰, C(═NR¹⁸)NR¹⁹R²⁰, C(═NOR¹⁸)NR¹⁹R²⁰ or R²¹,

wherein,

R¹⁸, R¹⁹ and R²⁰ independently is H, alkyl, alkenyl, alkynyl, alkadienyl, cycloalkyl, cycloheteroalkyl, aryl or heteroaryl, optionally substituted with one or more substituents selected from the group consisting of halogen, CN, CNO, C(halogen)₃, OR²¹, OC(═O)R²¹, OC(═O)OR²¹, OC(═O)NR²¹R²², SR²¹, S(═O)R²¹, S(═O)₂R²¹, S(═O)₂NR²¹R²², NO₂, N₃, NR²¹R²², N⁺R²¹R²²R²³, NR¹⁸NR¹⁹R²⁰, NR²¹C(═O)R²², NR²¹C(═O)OR²², NR²¹C(═O)NR²²R²³, NC, P(═O)(OR²¹)OR²², P³⁰R¹⁸R¹⁹R²⁰, C(═O)R²¹, C(═NR²¹)R²², C(═NOR²¹)R²², C(═NNR²¹R²²), C(═O)OR²¹, C(═O)NR²¹R²², C(═O)NR²¹OR²²C(═NR¹⁸)NR¹⁹R²⁰C(═NOR¹⁸)NR¹⁹R²⁰ or C(═O)NR²¹NR²²R²³, wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

wherein,

R²¹, R²² and R²³ independently is H, alkyl, alkenyl, alkynyl, alkadienyl, cycloalkyl, cycloheteroalkyl, aryl or heteroaryl and wherein R²¹ and R²² may together form a 3-8 membered heterocyclic ring or R²¹ and R²³ may together form a 38 membered heterocyclic ring or R²² and R²³ may together form a 3-8 membered heterocyclic ring,

In a further embodiment,

R¹⁷ and R²⁴ independently is H, C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl or heteroaryl, optionally substituted with one or more substituents selected from the group consisting of SnR¹⁸ R¹⁹,R²⁰, Sn(OR¹⁸)R¹⁹R²⁰, Sn(OR¹⁸)(OR¹⁹)R²⁰, BR¹⁸R¹⁹, B(OR¹⁸)R¹⁹, B(OR¹⁸)(OR¹⁹), halogen, CN, CNO, C(halogen)₃, OR¹⁸, OC(═O)R¹⁸, OC(═O)OR¹⁸, OC(═O)NR¹⁸R¹⁹, SR¹⁸, S(═O)R¹⁸, S(═O)₂R¹⁸, S(═O)₂NR¹⁸R¹⁹, NO₂, N₃, NR¹⁹R¹⁹, N⁺R¹⁸R¹⁹R²⁰, NR¹⁸OR¹⁹, NR¹⁸NR¹⁹R²⁰, NR¹⁸C(═O)R¹⁹, NR¹⁸C(═O)OR¹⁹, NR¹⁸C(═O)NR¹⁹R²⁰, NC, P(═O)(OR¹⁸)OR₁₉, P⁺R¹⁸R¹⁹R²⁰, C(═O)R¹⁸, C(═NR¹⁸)R¹⁹, C(═NOR¹⁸)R¹⁹, C(═NNR¹⁸R¹⁹), C(═O)OR¹⁸, C(═O)NR¹⁸R¹⁹, C(═O)NR¹⁸OR¹⁹, C(═O)NR¹⁸NR¹⁹R²⁰, C(═NR¹⁸)NR¹⁹R²⁰, C(═NOR¹⁸)NR¹⁹R²⁰ or R²¹,

wherein,

R¹⁸, R¹⁹, R²⁰ and R²¹ independently is H, C₁-C₆ alkyl, C₂-C₈ alkenyl, C₂-C₆ alkynyl, C₄-C₈ alkadienyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In another embodiment,

R¹⁷ and R²⁴ independently is H, C₁-C₆ alkyl, C₃-C₇ cycloalkyl, C₃-C₇ cycloheteroalkyl, aryl or heteroaryl, optionally substituted with one or more substituents selected from the group consisting of halogen, CN, C(halogen)₃, OR¹⁸, OC(═O)R¹⁸, OC(═O)OR¹⁸, OC(═O)NR¹⁸R¹⁹, SR¹⁸, S(═O)R¹⁸, S(═O)₂R¹⁸, S(═O)₂NR¹⁸R¹⁹, NO₂, NR¹⁸R²⁰, NR¹⁸OR¹⁹, NR¹⁸NR¹⁹R²⁰, NR¹⁸C(═O)R¹⁹, NR¹⁸C(═O)OR¹⁹, NR¹⁸C(═O)NR¹⁹R²⁰, P(═O)(OR¹⁸)OR¹⁹, C(═O)R¹⁸, C(═NR¹⁸)R¹⁹, C(═NOR¹⁸)R¹⁹, C(═NNR¹⁸R¹⁹), C(═O)OR¹⁸, C(═O)NR¹⁸R¹⁹, C(═O)NR¹⁸OR¹⁹, C(═O)NR¹⁸NR¹⁹R²⁰, C(═NR¹⁸)NR¹⁹R²⁰, C(═NOR¹⁸)NR¹⁹R²⁰ or R²¹,

wherein,

R¹⁸, R¹⁹, R²⁰ and R²¹ independently is H, C₁-C₆ alkyl, C₃-C₇ cycloalkyl, C₃-C₇ cyclo-heteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, C₁-C₆ alkyl, C₃-C₇ cycloalkyl, C₃-C₇ cycloheteroalkyl, aryl or heteroaryl, optionally substituted with one or more substituents selected from the group consisting of F, Cl, CN, CF₃, OR¹⁸, OC(═O)R¹⁸, OC(═O)OR¹⁸, OC(═O)NR¹⁸R¹⁹, SR¹⁸, S(═O)R¹⁸, S(═O)₂R¹⁸, S(═O)₂NR¹⁸R¹⁹, NO₂, NR¹⁸R¹⁹, NR¹⁸OR¹⁹, NR¹⁸NR¹⁹R²⁰, NR¹⁸C(═O)R¹⁹, NR¹⁸C(═O)OR¹⁹, NR¹⁸C(═O)NR¹⁹R²⁰, P(═O)(OR¹⁸)OR¹⁹, C(═O)R¹⁸, C(═NR¹⁸)R¹⁹, C(═NOR¹⁸)R¹⁹, C(═NNR¹⁸R¹⁹), C(═O)OR¹⁸, C(═O)NR¹⁸R¹⁹, C(═O)NR¹⁸OR¹⁹, C(═O)NR¹⁸NR¹⁹R²⁰, C(═NR¹⁸)NR¹⁹R²⁰, C(═NOR¹⁸)NR¹⁹R²⁰ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, C₁-C₆ alkyl, C₃-C₇ cycloalkyl, C₃-C₇ cycloheteroalkyl, aryl or heteroaryl, optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, methyl, ethyl, propyl, butyl, cyclopropyl, cyclobutyl, cyclopentyl, cyclohexyl, aziridinyl, azetidinyl, pyrrolidinyl, piperidinyl, morpholinyl, phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)OR¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, N¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸R¹⁹ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁸, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, C₁-C₆ alkyl, C₃-C₇ cycloalkyl, C₃-C₇ cycloheteroalkyl, aryl or heteroaryl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁶, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁶OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl, butyl, cyclopropyl, cyclobutyl, cyclopentyl, cyclohexyl, phenyl, naphthyl, thienyl, furyl, pyridinyl, quinolinyl or isoquinolinyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁹C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R₁₉, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is H, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, methyl, ethyl, propyl or butyl and wherein R¹⁸ and R¹⁹ may together form a 3-8 membered heterocyclic ring or R¹⁸ and R²⁰ may together form a 3-8 membered heterocyclic ring or R¹⁹ and R²⁰ may together form a 3-8 membered heterocyclic ring,

In still another embodiment,

R¹⁷ and R²⁴ independently is methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl,

In still another embodiment,

R¹⁷ and R²⁴ independently is aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl,

In still another embodiment,

R¹⁷ and R²⁴ independently is phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl,

In still another embodiment,

R¹⁷ and R²⁴ independently is phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl,

In still another embodiment,

R¹⁷ and R²⁴ independently is thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, cyclopropyl, cyclobutyl, cyclopentyl or cyclohexyl,

In still another embodiment,

R¹⁷ and R²⁴ independently is methyl, ethyl, propyl, butyl, cyclopropyl, cyclobutyl, cyclopentyl or cyclohexyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C)═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R₁₈, R¹⁹, R²⁰ and R²¹ independently is H, phenyl, naphthyl, thienyl, furyl, pyridinyl, quinolinyl or isoquinolinyl,

In still another embodiment,

R¹⁷ and R²⁴ independently is aziridinyl, azetidinyl, pyrrolidinyl, piperidinyl or morpholinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl,

In still another embodiment,

R¹⁷ and R²⁴ independently is phenyl, naphtyl, thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl,

In still another embodiment,

R¹⁷ and R²⁴ independently is phenyl or naphtyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl,

In still another embodiment,

R¹⁷ and R²⁴ independently is thienyl, furyl, pyridyl, quinolinyl or isoquinolinyl optionally substituted with one or more substituents selected from the group consisting of F, Cl, CN, CF₃, OR¹⁸, S(═O)R¹⁸, S(═O)₂R¹⁸, S(═O)₂NR¹⁸R¹⁹, NO₂, NR¹⁸R¹⁹, NR¹⁸C(═O)R¹⁹, NR¹⁸C(═O)OR¹⁹, NR¹⁸C(═O)NR¹⁹R²⁰, C(═O)R¹⁸, C(═NOR¹⁸)R¹⁹, C(═O)OR¹⁸, C(═O)NR¹⁸R¹⁹, C(═O)NR¹⁸OR¹⁹ or R²¹,

wherein,

R¹⁸, R¹⁹, R²⁰ and R²¹ independently is H, phenyl, naphthyl, thienyl, furyl, pyridinyl, quinolinyl or isoquinolinyl,

In still another embodiment,

R¹⁷ and R²⁴ independently is H, C₁-C₆ alkyl, C₃-C₇ cycloalkyl, C₃-C₇ cycloheteroalkyl, aryl or heteroaryl

In still another embodiment,

R¹⁷ and R²⁴ independently is H,

In still another embodiment,

R¹⁷ and R²⁴ independently is C₁-C₆ alkyl, C₃C₇ cycloalkyl or C₃-C₇ cycloheteroalkyl,

In still another embodiment,

R¹⁷ and R²⁴ independently is methyl, ethyl, propyl or butyl

In still another prefered embodiment

R¹⁷ and R²⁴ independently is cyclopropyl, cyclobutyl, cyclopentyl or cyclohexyl

In still another prefered embodiment

R¹⁷ and R²⁴ independently is aziridinyl, pyrrolidinyl, piperidinyl or morpholinyl

In still another embodiment,

R¹⁷ and R²⁴ independently is aryl or heteroaryl

In still another embodiment,

R¹⁷ and R²⁴ independently is phenyl or naphthyl

In still another embodiment,

R¹⁷ and R²⁴ independently is thienyl, furyl, pyridyl, quinolinyl or isoquinolyl

Experiments

General Procedure 1: Synthesis of Benzoic Acid Derivatives for Building Blocks:

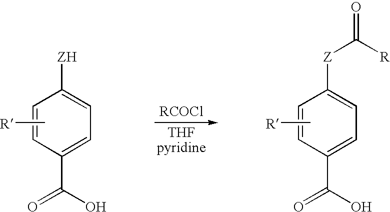

The benzoic acid derivative (1 mmol) was dissolved in THF (5 mL) and pyridine (3 mmol). The mixture was cooled to 0° C. and treated with an acid chloride (1.2 mmol). The cooling bath was removed and the reaction mixture was stirred for 1 hour at rt. Toluene (10 mL) was added and the solution was evaporated in vacuo. The crude was redissolved in EtOAc (10 mL), washed with water and brine. The organic phase was dried over MgSO₄ and evaporated in vacuo. The pure product was obtained by silica gel purification using a gradient of heptane to EtOAc as eluent.

### EXAMPLE 1

### General Procedure 1, wherein Z=S, R′═H, and R═CH₃

### 4-Acetylsulfanyl-benzoic acid

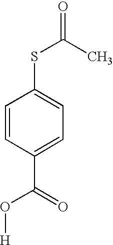

Yield=70%: ¹H-NMR (DMSO-d₆): 8.00 (d, 2H); 7.55 (d, 2H); 2.46 (s, 3H).

### EXAMPLE 2

### General Procedure 1, wherein Z=S, R′═H, and R═CH₂CH₃

### 4Propionylsulfanyl-benzoic acid

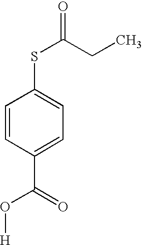

Yield=85%: ¹H-NMR (CDCl₃): 8.12 (d, 2H); 7.58 (d, 2H); 2.76 (q, 2H); 1.28 (t, 3H).

### EXAMPLE 3

### General Procedure 1, wherein Z=S, R′═H, and R═(CH₂)₂CH₃

### 4-Butyrylsulfanyl-benzoic acid

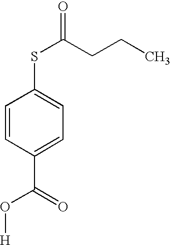

Yield=98%: ¹H NMR (CDCl₃): 8.15 (d, 2H); 7.56 (d, 2H); 2.70 (t, 2H); 1.81 (sixtet, 2H); 1.04 (t, 3H).

### EXAMPLE 4

### General Procedure 1, wherein Z=S, R′═H, and R═(CH₂)₂CHCH₂

### 4-Pent-4-enoylsulfanyl-benzoic acid

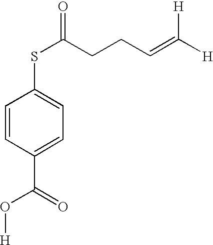

Yield=71%: ¹H-NMR (CDCl₃): 8.15 (d, 2H); 7.55 (d, 2H); 5.85 (m, 1H); 5.11 (dd, 2H); 2.82 (t, 2H); 2.47 (q, 2H).

### EXAMPLE 5

### General Procedure, wherein Z=O, R′═Cl, and R═CH₃

### 4-Acetoxy-3-chloro-benzoic acid

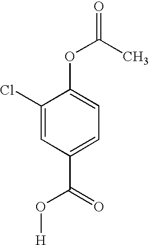

Yield=95%: ¹H nmr (CDCl₃): 8.20 (d, 1H); 8.05 (dd, 1H), 7.25 (d, 1H); 2.40 (s, 3H).
General Procedure 2: Synthesis of Nicotinic Acid Derivative for Building Blocks:

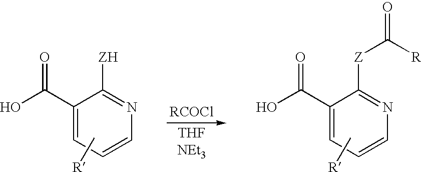

The nicotinic acid derivative (6.44 mmol) was dissolved in THF (10 mL) and triethyl-amine (5 mL). The mixture was cooled to 0° C. and treated with an acid chloride (12.88 mmol). The cooling bath was removed and the reaction mixture was stirred overnight at rt. After removal of the solvents, toluene (10 mL) was added to the crude and evaporated in vacuo. The pure product was obtained by silica gel purification using a gradient starting from dichloromethane going to 2% methanol in dichloromethane as eluent.

### EXAMPLE 6

### General Procedure 2, wherein Z=S, R′═H, and R═CH₃

### 2-Acetylsulfanyl-nicotinic acid

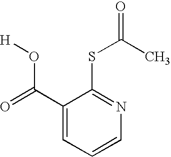

Yield=5%: ¹H-NMR (CDCl₃): 8.76 (dd, 1H); 8.64 (dd, 1H); 7.40 (dd, 1H); 2.79 (s, 3H).
General Procedure 3: Preparation of Building Blocks by Loading a Carrier-Functional Entity Ensemble Onto an Oligonucleotide Comprising an Amino Group:

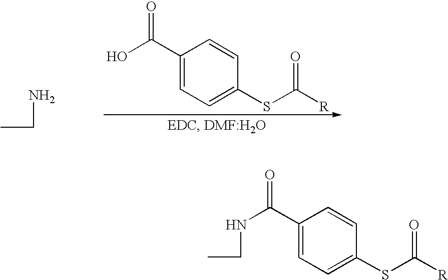

25 μL of a 150 mM benzoic acid derivative in DMF was mixed with 25 μL of a 150 mM solution of EDC in DMF. The mixture was left for 30 min at 25° C. 50 μL of an aminooligo (10 nmol) in 100 mM HEPES buffer pH 7.5 was added and the reaction mixture was left for 20 min at 25° C. The excess building block was removed by extraction with EtOAc (500 μL) and remaining EtOAc was removed in vacuo by spinning 10 min in a speedvac. The aminooligo loaded with the benzoic acid derivative was ethanol precipitated twice using NH₄OAc and analysed by electron spray mass spectrometry (ES-MS).

Aminooligo's used:

A: 5′-XTTTTTTTTTTTTTTTACGACTACGTTCAGGCAAGTB
B: 5′-XTTTTTTTTTTTTTTTTTTTTACGACTACGTTCAGGCAAGTB
C: 5′-XTTTTTTTTTTTTTTTTTTTTTTTTTACGACTACGTTCAGGCAA
GTB
D: 5′-BGACCTGTCGAGCATCCAGCZ
E: 5′-BGCATCCATCGY
X = 5′ amino C6 (Glen# 10-1906-90)
Y = C2 amino cIT phosphate (Glen# 10-1037-90)
Z = C6 amino dT phosphate (Glen# 10-1039)
B = Biotin (Glen# 10-1953-95)

### EXAMPLE 7

### General Procedure (3)

Oligo A loaded with compound of Example 1

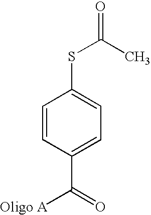

MS (calc., M-1)=11.560,87; MS (found)=11.557,89

### EXAMPLE 8

### General Procedure (3)

Oligo B loaded with compound of Example 1

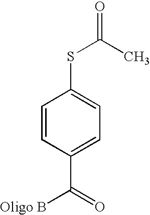

MS (calc., M-1)=13.081,87; MS (found)=13.079,01

### EXAMPLE 9

### General Procedure (3)

Oligo C loaded with compound of Example 1

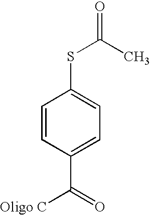

MS (calc., M-1)=14.602,86; MS (found)=14.599,66

### EXAMPLE 10

### General Procedure (3)

Oligo D loaded with compound of Example 1

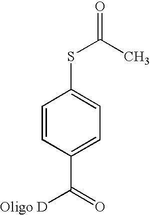

MS (calc., M-1)=6892,85; MS (found)=6893,29

### EXAMPLE 11

### General Procedure (3)

Oligo E loaded with compound of Example 1

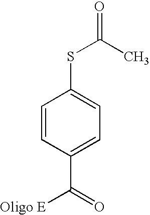

MS (calc., M-1) 4052,05; MS (found)=4067,49¹
¹ The difference observed in the calculated and found MS of around 16 is probably due to an oxidation of the sulphur atom of the biotin moiety

### EXAMPLE 12

### General Procedure (3)

Oligo E loaded with compound of Example 5

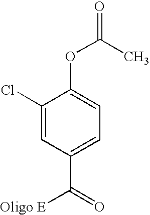

MS (calc., M-1)=4069,84; MS (found)=4070,20
General Procedure 4: Preparation of Building Blocks by Step Wise Loading of a Carrier and a Functional Entity Onto an Oligonucleotide Containing a Nucleotide Derivative Comprising an Amino Group:

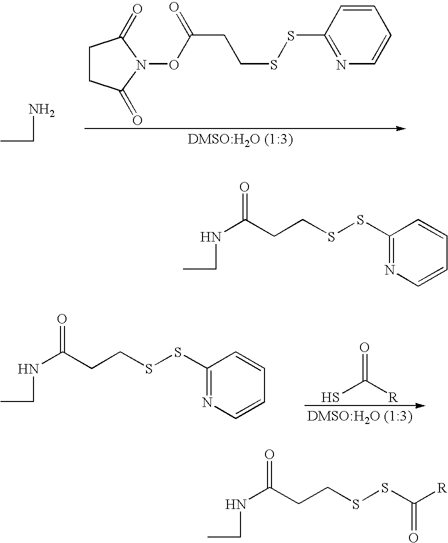

40 μL of a 20 mM SPDP solution in DMSO was mixed with an aminooligo (5 nmol). 200 mM HEPES buffer pH 7.5 was added (80 μL) and water to a final volume of 160 μL. the reaction mixture was left for 2 hours at 30° C. The excess building block was removed by extraction with EtOAc (500 μL). Remaining EtOAc was removed in vacuo by spinning 10 min in a speedvac. The SPDP activated aminooligo was purified using a micro bio-spin column (equilibrated with 200 mM HEPES buffer pH 7.5). 10 μL of a 50 mM thio acid derivate solution in DMSO was added to the purified SPDP activated aminooligo solution and the reaction mixture was left for 30 min at 20° C. The building block loaded aminooligo was ethanol precipitated twice using NH₄OAc and analysed by electron spray mass spectrometry (ES-MS).

Aminooligo used:

A2:
5′-GACCTGTCGAGCATCCAGCTTCATGGGAATTCCTCGTCCACAATGZ

Z=Amino-Modifier C6 dT phosphate (Glen# 10-1039-)

### EXAMPLE 13

### General Procedure (4)

Oligo A2 loaded with thiobenzoic acid

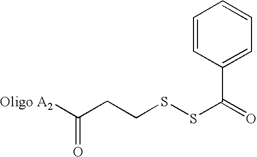

MS (calc., M-1)=14518,76; MS (found)=14516,78

### EXAMPLE 14

### Loading of a Trisamine Scaffold on an Oligonucleotide Containing a Nucleotide Derivative Comprising an Amino Group:

A hexameric scaffold peptide with the sequence, CysPhePheLysLysLys, was synthesised by standard solid-phase Fmoc peptide chemistry. The scaffold peptide comprises a —SH group on the cystein side chain, said —SH group being used for coupling the scaffold peptide to a amine-bearing oligonucleotide serving as anticodon and linker. Each of the three lysin moieties comprises an amino group in the side chain. The amine groups are used as reactive groups for the formation of a connection to functional entities emanating from building blocks.

The N-terminus of the peptide was acetylated and the C-terminus was initially capped as an amide to avoid any participation in the reactions to follow and subsequently purified by reverse phase-HPLC. The scaffold peptide was covalently attached to DNA oligonucleotide using the scheme shown schematically below. For illustrative purposes, the scaffold is indicated as HSˆScaffold

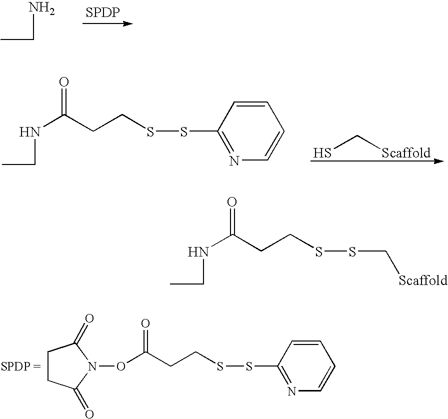

5 nmol of oligodeoxynucleotide F: 5′-XTCGTAACGACTGAATGACGT where X=5′ amino C6 (Glen# 10-1906-90) in 100 mM Hepes-OH pH 7.5 is incubated with 20 mM Succinimidyl-propyl-2-dithiopyridyl (SPDP, Molecular probes) dissolved in DMSO for 3 hours at 25° C. Excess SPDP is removed by triple extraction using 5 volumes of ethylacetate. The sample is further purified using a Bio-rad Microspin 6 column equilibrated in H₂O.

The oligonucleotide-scaffold conjugate is synthesised by incubating 1 μmol hexapeptide with 5 nmol SPDP activated oligonucleotide in 100 mM Hepes-OH pH 7.5 for 2 hours at 25° C. Excess peptide is removed by double sodium-acetate/ethanol precipitation of the scaffold-DNA complex according to standard procedure. The loading was verified by Electrospray Mass Spectrometry (ES-MS).

Loading of trisamine scaffold on oligo F: MS (calc., M-1)=7247.45 MS (found)=7244.80

### EXAMPLE 15

### Transfer of a Functional Entity from a Building Block to a Scaffold:

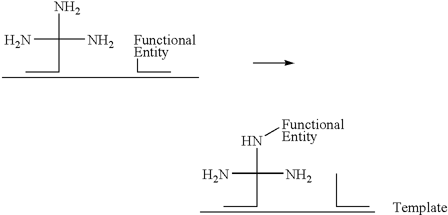

A template oligo G: 5′-ACGTCATTCAGTCGTTACGAACGATGGATGCTCCAGG TCGC (1 nmol) was mixed with scaffold oligo F (1.5 nmol) in MES-buffer (20 μL of a 100 mM MES, pH=6) and water (added to a final volume of 100 μL). Scaffold oligo F was annealed to the template by heating to 80° C. and cooled (−2° C./10 second) to room temperature and functional entity oligo E (Example 11) (1.5 nmol) was added. The mixture was left o/n at room temperature. The oligo complex was attached to streptavidine by addition of streptavidine sepharose beads (50 μL, prewashed with 2×1 mL 100 mM MES buffer, pH=6). The beads were washed with water (4×200 μL). Oligo F was separated from the streptavidine bound complex by addition of water (200 uL) followed by heating to 80° C. for 5 minute. The beads were filtered off and the water was evaporated. Oligo F was redissolved in water and building block transfer verified by electron spray mass spectrometry (ES-MS).

Transfer of acetyl to trisamine scaffold oligo F from example I attached to oligo E: MS (calc.)=7289.49; MS (found)=7286.58

Section 3: Transfer Efficiencies of Functional Entities from Building Blocks to Amine Scaffolds

Carrier coupled functional entities were loaded onto oligos (oligonucleotides) containing a nucleotide derivative comprising an amino group (General procedure 5) or a nucleotide derivative comprising a thiol (General procedure 6) and the transfer was conducted to a scaffold oligo with a nucleotide derivative comprising an amino group. Transfer efficiencies were analyzed by ES-MS (electrospray mass spectroscopy) (General procedure 7).

General Procedure 5: Loading of a Carrier Coupled Functional Entity onto an Amino Oligo

25 μl 100 mM carrier coupled functional entity dissolved in DMF (dimethyl formamide) was mixed with 25 μl 100 mM EDC (1-ethyl-3-(3-dimethylaminopropyl) carbodiimide hydrochloride) in DMF for 30 minutes at 25° C. The mixture was added to 50 μl amino oligo in H₂O with 100 mM HEPES (2-[4(2-hydroxy-ethyl)-piperazin-1-yl]-ethanesulfonic acid) pH 7.5 and the reaction was allowed to proceed for 20 minutes at 25° C. Unreacted carrier coupled functional entity was removed by extraction with 500 μl EtOAc (ethyl acetate), and the oligo was purified by gel filtration through a microspin column equilibrated with 100 mM MES (2-(N-morpholino) ethanesulfonic acid) pH 6.0.

Oligonucleotide used:

Oligo G: 5′-GCGACCTGGAGCATCCATCGY

Y=Amino-Modifier C2 dT phosphate (Glen# 10-1037)

### EXAMPLE 16

### General Procedure 5, Using Compound of Example 5 as Carrier Coupled Functional Entity

Carrier coupled functional entity: 4-Acetoxy-3-chloro-benzoic acid

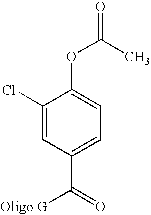

Mass: 6738.23 (observed using ES-MS), 6738.31 (calculated) (The carrier coupled functional entity oligo is hydrolyzed in the mass spectrometer during analysis).

### EXAMPLE 17

### General Procedure 5, Using Compound of Example 1 as Carrier Coupled Functional Entity

Carrier coupled functional entity: 4-Acetylsulfanyl-benzoic acid

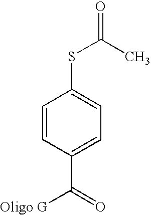

Mass: 6718.48 (observed using ES-MS), 6719.48 (calculated) (The carrier coupled functional entity oligo is hydrolyzed in the mass spectrometer during analysis).

### EXAMPLE 18

### General Procedure 1, wherein Z=O, R′═NO₂, and R═CH₃ and General Procedure 5

Carrier coupled functional entity: 4-Acetoxy-3-nitro-benzoic acid

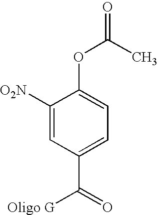

Mass: 6748.31 (observed using ES-MS), 6748.42 (calculated) (The carrier coupled functional entity oligo is hydrolyzed in the mass spectrometer during analysis).

General Procedure 6: Loading of a Carrier Coupled Functional Entity Onto a Thiol Oligo:

10 nmol thiol oligo was lyophilized and redissolved in 50 μl H₂O with 100 mM dithiothretiol and 100 mM sodium phosphate pH 8.0 and incubated at 37° C. for 1 hour. The reduced oligo was purified using a microspin column equilibrated with HEPES (100 mM, pH 7.5). Then 100 mM NHM (N-hydroxymaleimide) in HEPES (100 mM, pH 7.5) was added to the thiol oligo and the mixture was incubated at 25° C. for 2 hours. The resulting NHS (N-hydroxysuccinimide)-oligo was purified using a microspin column equilibrated with H₂O. 1 nmol NHS-oligo was lyophilized and redissolved in 10 μl 100 mM MES, pH 6. 50 μl carrier coupled functional entity (100 mM) in dimethyl formamide was activated with 50 μl 100 mM EDC in DMF for 30 min at 25° C. 10 μl of the EDC-activated carrier coupled functional entity was mixed with the NHS-oligo and incubated for 5 min at 25 ° C. 30 μl 100 mM MES pH 6 was added and following an extraction with 500 μl EtOAc the oligo was purified using a microspin column equilibrated with 100 mM MES pH6.

Oligo H: 5′-GCGACCTGGAGCATCCATCGTX

X=Thiol-Modifier C6 S—S (Glen# 10-1936)

### EXAMPLE 19

### General Procedure 6

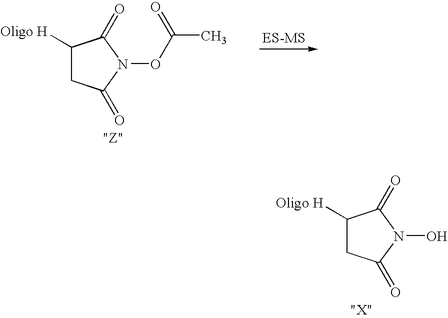

Mass “X”: 6723.21 (observed using ES-MS), 6723.52 (calculated) (Compound “Z” is hydrolyzed to compound “X” in the mass spectrometer during analysis).
General Procedure 7: Transfer of Functional Entity from a Carrier Oligo to a Scaffold Oligo.

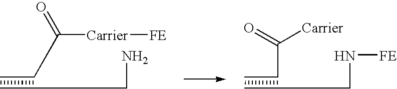

Scaffold oligo I: 5′-ZACGATGGATGCTCCAGGTCGC

Z=5′ Amino-modifier C6 (Glen Research cat. # 10-1906)

A carrier coupled functional entity oligo (Examples 16, 17, 18, 19) (250 pmol) was added to a scaffold oligo I (200 pmol) in 50 μl 100 mM MES, pH 6. The mixture was incubated overnight at 25° C. Subsequently, the mixture was purified by gel filtration using a microspin column equilibrated with H₂O and transfer of the functional entity was verified by electron spray mass spectrometry (ES-MS). Transfer efficiencies are expressed in percent and were calculated by dividing the abundance of scaffold oligo carrying transferred functional entities to total abundance of scaffold oligos (with and without transferred functional entities).

### EXAMPLE 20

### General Procedure 7

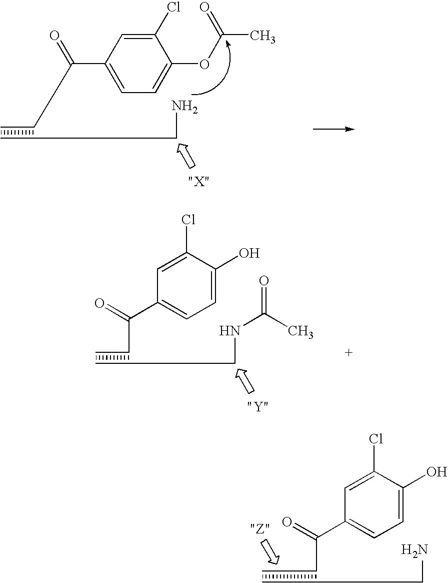

Mass (“X”): 6624.70 (observed), 6625.42 (calculated). Abundance: 73.16 (arbitrary units)

Mass (“Y”): 6666.09 (observed), 6667.46 (calculated). Abundance: 26.15 (arbitrary units)

Mass (“Z”): 6738.01 (observed), 6738.31 (calculated) (carrier coupled functional entity oligos are hydrolyzed in the mass spectrometer during analysis).

Transfer efficiency calculated as: 26.15/(26.15+73.16)=0.2633˜26%

Transfer efficiencies:

| Scaf- | | |
|---|---|---|
| fold | | |
| oligo | Building block oligo | |
| | 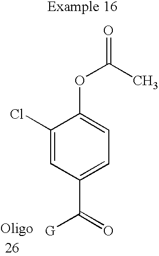 | 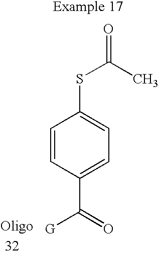 |
| | 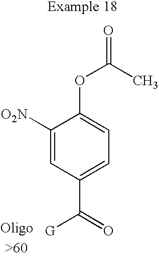 | 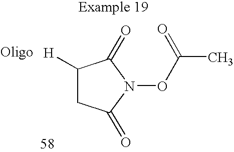 |

### EXAMPLE 21

### Stability of Building Block Oligonucleotides During Storage and Handling

Carrier coupled functional entities were loaded onto oligonucleotides containing a nucleotide derivative comprising an amino group (General Procedure 7). The resulting carrier coupled functional entity oligos were either mixed immediately with scaffold oligo I at 25° C. (condition 1) or subjected to different conditions before mixing: (condition 2) −80° C. for 14 days, (condition 3) 25° C. for 1 hour. For condition 4 the scaffold oligo and the building block oligo were heated to 80° C. for 30 seconds, mixed, and then cooled to 25° C. (−5° C./minute). The functional entity of the building block oligo was transferred to a scaffold oligo by incubation at 25° C. overnight and analyzed by ES-MS (General procedure 3).

Transfer efficiencies (in percent) in reactions involving the same building block were normalized to facilitate comparison, e.g. the observed transfer efficiency when scaffold oligo was mixed with building block oligo immediately after production was set to 100:

| | | Ex. | Ex. | Ex. | Ex. |
|---|---|---|---|---|---|
| Condition | Description | 16 | 17 | 18 | 19 |
| 1 | Immediate mixing | 100 | 100 | 100 | 100 |
| 2 | −80° C. for 14 days | 96 | 97 | 89 | 92 |
| 3 | 25° C. for 1 hour | 97 | 98 | 93 | 71 |
| 4 | From 80° C. to 25° C. | 106 | 105 | 87 | 60 |

The results indicate that all the building blocks may be stored in a freezer at −80° C. for several weeks without loosing significant reactivity. Under practical handling conditions at room temperature the NHS ester of example 19, which is not according to the invention, looses a considerable amount of reactivity. The tendency of spontaneous hydrolysis of the building block according to example 18 is reinforced under the condition simulating an actual experiment (condition 4), while the building blocks of example 16 to 18 have an acceptable stability or even a slightly increased activity. Activities above 100 observed under condition 4 might be due to experimental variation or facilitation of annealing of the carrier coupled functional entity oligo and scaffold oligo at elevated temperatures.

### EXAMPLE 22

### Preparation of Building Blocks

The following oligo containing a nucleobase modified with an amino group was synthesised, using the conventional phosphoramidite approach:

N: 5′-ZGT AAC ACC TGT GTA AGC TGC CTG TCA GTC GGT
ACT GAC CTG TCG AGC ATC CAG CT

Z depicts the nucleobase modified with an aminogroup, incorporated using the commercially available amino modifier C6 dT phosphoramidite (10-1039-90 from Glen research)

The loading with a functional entity proceeds using the general method: An amino oligo (3 pmol) was mixed with a phosphate buffer (3 uL of a 0.1 M solution, pH═6) and NaBH₃CN (3 uL of a 1 M solution in MeOH). A chemical entity comprising the functional entity (3 uL of a 1 M solution in MeOH) was added and the mixture was left o/n at room temperature. The product formation was analysed by PAGE gel.

Exemplary chemical entities are 4-acetoxybenzaldehyde (24,260-8 from Sigma-Aldrich),

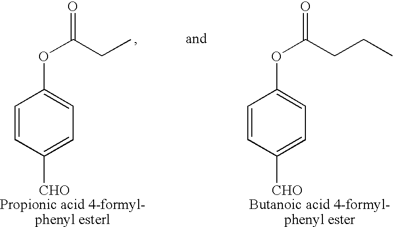

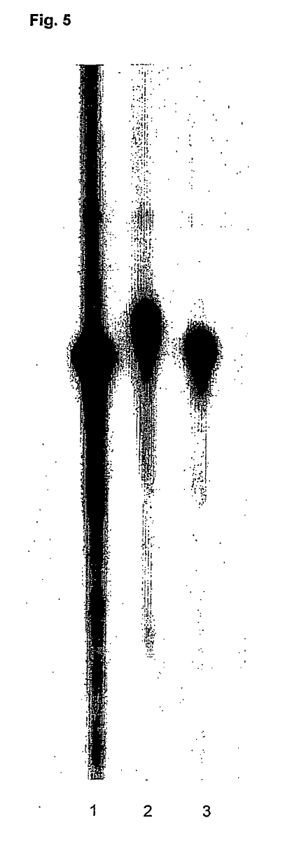

FIG. 5 shows a PAGE analysis of the loading of an oligo with butanoic acid 4-formyl-phenyl ester. Lane 1 shows the reference amino oligo (N). Lane 2 show the amino oligo (N) after loading with a the chemical entity comprising the functional entity, and Lane 3 shows removal of the functional entity, attached in lane 2, by treatment with pH=11 for 1 hour.

The above examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full content of this document, including the examples shown above and the references to the scientific a patent literature cited herein. It should further be appreciated that the contents of those cited references are incorporated herein by reference to help illustrate the state of the art. The examples above contain important additional information that can be adapted to the practice of this invention in its various embodiments and the equivalents thereof.

| | |
|---|---|
| DCC | N,N′-Dicyclohexylcarbodiimide |
| DhbtOH | 3,4-dihydro-3-hydroxy-4-oxo-1,2,3-benzotriazine |
| DIC | Diisopropylcarbodiimide |
| DIEA | Diethylisopropylamin |
| DMAP | 4-Dimethylaminopyridine |
| DNA | Deoxyribosenucleic Acid |
| EDC | 1-Ethyl-3-(3′-dimethylaminopropyl)carbodiimide-HCl |
| HATU | 2-(1H-7-Azabenzotriazole-1-yl)-1,1,3,3-tetramethyluronium |
| | hexafluorophosphate |
| HBTU | 2-(1H-Benzotriazole-1-yl)-1,1,3,3-tetramethyluronium |
| | hexafluorophosphate |
| HOAt | N-Hydroxy-7-azabenzotriazole |
| HOBt | N-Hydroxybenzotriazole |
| LNA | Locked Nucleic Acid |
| NHS | N-hydroxysuccinimid |
| OTf | Trifluoromethylsulfonate |
| OTs | Toluenesulfonate |
| PNA | Peptide Nucleic Acid |
| PyBoP | Benzotriazole-1-yl-oxy-tris-pyrrolidino-phosphonium |
| | hexafluorophosphate |
| PyBroP | Bromo-tris-pyrrolidino-phosphonium hexafluorophosphate |
| RNA | Ribonucleic acid |
| TBTU | 2-(1H-Benzotriazole-1-yl)-1,1,3,3-tetramethyluronium |
| | tetrafluoroborate |
| TEA | Triethylamine |
| RP-HPLC | Reverse Phase High Performance Liquid Chromatography |
| TBDMS-Cl | Tert-Butyldimethylsilylchloride |
| 5-Iodo-dU | 5-iodo-deoxyriboseuracil |
| TLC | Thin layer chromatography |
| (Boc)₂O | Boc anhydride, di-tert-butyl dicarbonate |
| TBAF | Tetrabutylammonium fluoride |
| SPDP | Succinimidyl-propyl-2-dithiopyridyl |

1. A building block of the general formula
Complementing Element—Linker—Carrier—C—F-connecting group—Functional entity precursor
capable of transferring a functional entity to a recipient reactive group, wherein
Complementing Element is a group identifying the functional entity precursor,
Linker is a chemical moiety comprising a Spacer and a S—C-connecting group, wherein the Spacer is a valence bond or a group distancing the functional entity precursor to be transferred from the complementing element and the S—C-connecting group connects the spacer with the Carrier,
Carrier is selected among the groups 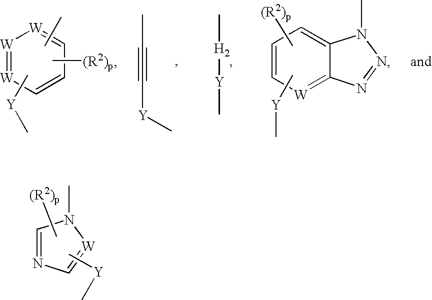
wherein the Linker attaches to the Carrier through Y and W═CH or N R²═H, -Halogen, —NO₂, —CN, —C(Halogen)₃, —C(O)R³, —C(O)NHR³, C(O)NR³₂, —NC(O)R³, —S(O)₂NHR³, —S(O)₂NR³₂, —S(O)₂R³, —P(O)₂—R³, —P(O)—R³, —S(O)—R³, P(O)—OR³, —S(O)—OR³, —N⁺R³₃, wherein p is an integer of 0 to 3, R³═H, C₁-C₆ alkyl, C₁-C₆ alkenyl, C₁-C₆ alkynyl, or aryl, and Halogen is F, Cl, Br, or I, Y=absent, C₁-C₆ Alkylene, C₁-C₆ Alkenylene, C₁-C₆ Alkynylene, Arylene, Heteroarylene, Carbonyl, or —SO₂CH₂—,
C—F-connecting group is 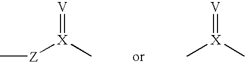
where the carrier is connected to the left hand side of the formulae and X═—C—, —S—, —P—, —S(O)— or —P(O)—, V═O, S, NH, or N—C₁-C₆ alkyl, and Z=O, S, and
Functional entity precursor is H or selected among the group consisting of a C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R⁴, 0-3 R⁵ and 0-3 R⁹, or selected among the group consisting of C₁-C₃ alkylene-NR⁴₂, C₁-C₃ alkylene-NR⁴C(O)R⁸, C₁-C₃ alkylene-NR⁴C(O)OR⁸, C₁-C₂ alkylene-O—NR⁴₂, C₁-C₂ alkylene-O—NR⁴C(O)R⁸, and C₁-C₂ alkylene-O—NR⁴C(O)OR⁸ substituted with 0-3 R⁹.
where R⁴ is H or selected independently among the group consisting of C₁-C₆ alkyl, C₂-C₆ alkenyl, C₂-C₆ alkynyl, C₃-C₇ cycloalkyl, C₃-C₇ cycloheteroalkyl, aryl, heteroaryl, said group being substituted with 0-3 R⁹ and
R⁵ is selected independently from —N₃, —CNO, —C(NOH)NH₂, —NHOH, —NHNHR⁶, —C(O)R⁶, —SnR⁶₃, —B(OR⁶)₂, —P(O)(OR⁶)₂ or the group consisting of C₂-C₆ alkenyl, C₂-C₆ alkynyl, C₄-C₈ alkadienyl said group being substituted with 0-2 R⁷,
where R⁶ is selected independently from H, C₁-C₆ alkyl, C₃-C₇ cycloalkyl, aryl or C₁-C₆ alkylene-aryl substituted with 0-5 halogen atoms selected from —F, —Cl, —Br, and —I; and R⁷ is independently selected from —NO₂, —COOR⁶, —COR⁶, —CN, —OSiR⁶₃, —OR⁶ and —NR⁶₂. R⁸ is H. C₁-C₆ alkyl, C₂-C₆ alkenyl, C₂-C₆ alkynyl, C₃-C₇ cycloalkyl, aryl or C₁-C₆ alkylene-aryl substituted with 0-3 substituents independently selected from —F, —Cl, —NO₂, —R³, —OR³, —SiR³₃ R⁹ is ═O, —F, —Cl, —Br, —I, —CN, —NO₂, —OR⁶, —NR⁶₂, —NR⁶—C(O)R⁸, —NR⁶—C(O)OR⁸, —SR⁶, —S(O)R⁶, —S(O)₂R⁶, —COOR⁶, —C(O)NR⁶₂ and —S(O)₂NR⁶₂.

2. The compound according to claim 1, wherein the Spacer is a valence bond, C₁-C₆ alkylene-A-, C₁-C₆ alkenylene-A-, C₂-C₆ alkynylene-A-, or 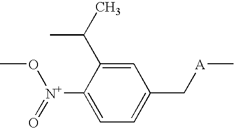
said spacer optionally being connected through A to a linker selected from 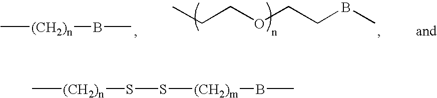
where A is —C(O)NR¹—, —NR¹—, —O—, —S—, or —C(O)—O—; B is —O—, —S—, —NR¹— or —C(O)NR¹— and connects to S—C-connecting group; R¹ is selected independently from H, C₁-C₆ alkyl, C₃-C₇ cycloalkyl, C₁-C₆ alkylene-aryl, or aryl substituted with 0-5 halogen atoms selected from —F, —Cl, —Br and —I; and n and m independently are integers ranging from 1 to 10.

3. The compound according to claim 1, wherein the Spacer is C₁-C₆ alkylene-A-, C₁-C₆ alkenylene-A-, C₂-C₆ alkynylene-A-, or 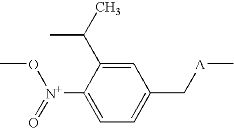
said spacer optionally being connected through A to a moiety selected from 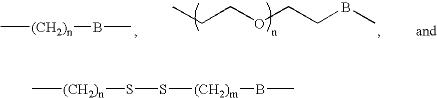
where A is —C(O)NR¹—, or —S—; B is —S—, —NR¹— or —C(O)NR¹— and connects to S—C-connecting group; R¹ is selected independently from H, C₁-C₆ alkyl, C₁-C₆ alkylene-aryl, or aryl; and n and m independently are integers ranging from 1 to 6.

4. The compound according to claim 1, wherein Spacer is -A-, a group C₁-C₆ alkylene-A-, C₂-C₆ alkenylene-A-, or C₂-C₆ alkynylene-A-optionally substituted with 1 to 3 hydroxy groups, or 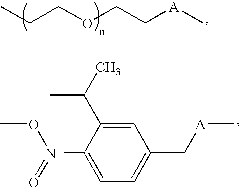
said spacer being connected through A to a linker selected from 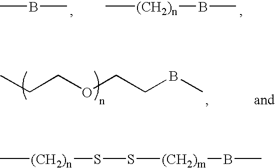
where A is a valence bond, —NR¹⁰—, —C(O)NR¹⁰—, —NR¹⁰—C(O)—, —O—, —S—, —C(O)—O— or —OP(═O)(O⁻)—O—; B is a valence bond, —O—, —S—, —NR¹⁰—, —C(O)— or —C(O)NR¹⁰— and connects to S—C-connecting group; R¹⁰ is selected independently from H, C₁-C₆ alkyl, C₃-C₇ cycloalkyl, aryl, C₁-C₆ alkylene-aryl, 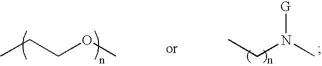
G is H or C₁-C₆ alkyl; and n and m independently are integers ranging from 1 to 10.

5. A compound according to claim 4, wherein the spacer is C₂-C₆ alkenylene-A, said spacer being connected through A to a moiety selected from 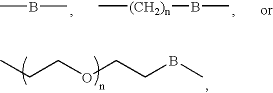
where A is a valence bond, —C(O)NR¹⁰—, —NR¹⁰—C(O)—, —S—, —C(O)—O— or —OP(═O)(O⁻)—O—; B is a valence bond, —S—, —NR¹⁰—, or —C(O)— and connects to S—C-connecting group; n and m independently are integers ranging from 1 to 10 and R¹⁰ is selected independently from H, 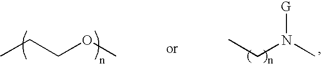
wherein G is H or C₁-C₆ alkyl; and the spacer is connected to the complementing element through a nucleobase.

6. A compound according to claim 4, wherein the spacer is -A-, 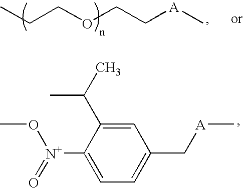
said spacer being connected through A to a moiety selected from 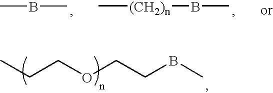
where A is a valence bond, —NR¹⁰—C(O)—, —O—, or —S—; B is a valence bond, —S—, —NR¹⁰—, or —C(O)— and connects to S—C-connecting group;
n and m independently are integers ranging from 1 to 10 and R¹⁰ is selected independently from H, 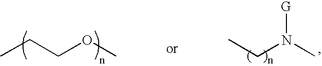
wherein G is H or C₁-C₆ alkyl; and the spacer is connected to the complementing element via a phosphorus group.

7. A compound according to claim 6, wherein the phosphorus group is a phosphate or thiophosphate group attached to a 3′ or 5′ end of a complementing element.

8. A compound according to claim 1, wherein the S—C-connecting group is a valence bond, —NH—C(═O)—, —NH—C(═O)—C₁-C₆ alkylene-, —S—S—, —S—S—C₁-C₆ alkylene-, —C₁-C₆ alkylene-S—S—, —C(═O)—NH—(C₁-C₆ alkylene)-, 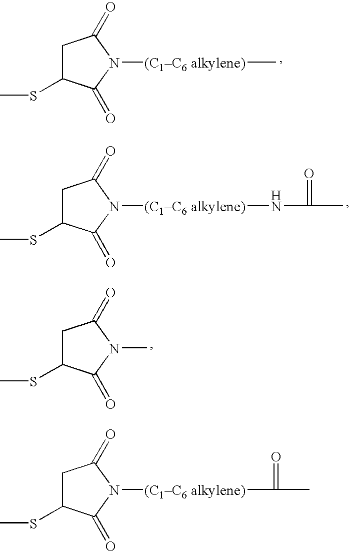
—NH—C(═O)-Arylene-C(R¹⁰)₂—NH—C(═O)—, —C(═O)—, —C(═O)—C₁-C₆ alkylene- or —C(═O)-Arylene-C(R¹⁰)₂—NR¹⁰—C(═O)—, where the right hand side of the formulae connects to the carrier.

9. A compound according claim 1, wherein the S—C-connecting group is a valence bond, —NH—C(═O)—, —NH—C(═O)—C₁-C₆ alkylene-, —S—S—, —S—S—C₁-C₆ alkylene-, —C(═O)—NH—(C₁-C₆ alkylene)-, 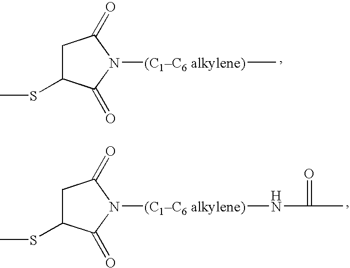
—NH—C(═O)-Arylene-C(R¹⁰)₂—NH—C(═O)—, where the right hand side of the formulae connects to the carrier.

10. A compound according to claim 1, wherein the S—C-connecting group is —S—S—, —C₁-C₆ alkylene-S—S—, —C(═O)—NH—(C₁-C₆ alkylene)-, —C(═O)—, or —C(═O)-Arylene-C(R¹⁰)₂—NR¹⁰—C(═O)—, where the right hand side of the formulae connects to the carrier.

11. A compound according to claim 1, wherein the S—C-connecting group is —S—S—, —C(═O)—, or —C(═O)-Arylene-C(R¹⁰)₂—NR¹⁰—C(═O)—, where the right hand side of the formulae connects to the carrier.

12. The compound according to claim 1, wherein the S—C-connecting group is a valence bond, —NH—C(═O)—, —S—S—, or —C(═O)—NH—, where the right hand side of the formulae connects to the carrier.

13. A compound according to claim 1, wherein the carrier is 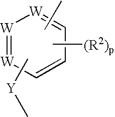
and attaches to the linker through Y, and W, Y, R², and p are as defined in claim 1.

14. A compound according to claim 1, wherein the carrier is 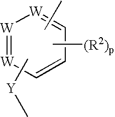
and attaches to the linker through Y and
W═CH
R²═H, halogen, —NO₂, —CN, —C(Halogen)₃, —C(O)R³, —C(O)NHR³, C(O)NR³₂, —S(O)₂NHR³, —S(O)₂NR³₂, —S(O)₂R³, —N⁺R³₃, wherein halogen is selected from the group consisting of —Cl, —F, —Br, and —I, p is an integer of 0 to 3, and R³═H, C₁-C₆ alkyl, or aryl,
Y=absent, C₁-C₆ Alkylene, or carbonyl.

15. A compound according to claim 1, wherein the C—F-connecting group is 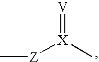
in which
Z=O, S
X═—C—, and
V═O.

16. A compound according to claim 1, wherein Complementing element is a nucleic acid.

17. A compound according to claim 1, wherein Complementing element is a sequence of nucleotides selected from the group of DNA, RNA, LNA PNA, morpholino derivatives, or combinations thereof.

18. A compound according to claim 1, wherein the Functional entity precursor is H or selected among the group consisting of a C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R⁵ and 0-3 R⁹, or selected among the group consisting of C₁-C₃ alkylene-NR⁴₂, C₁-C₃ alkylene-NR⁴C(O)R⁸, C₁-C₃ alkylene-NR⁴C(O)OR⁸, C₁-C₂ alkylene-O—NR⁴₂, C₁-C₂ alkylene-O—NR⁴C(O)R⁸, and C₁-C₂ alkylene-O—NR⁴C(O)OR⁸ substituted with 0-3 R⁹.

19. A compound according to claim 1, wherein the Functional entity precursor is H or selected among the group consisting of C₁-C₆ alkyl, C₂-C₆ alkenyl, C₂-C₆ alkynyl, C₄-C₈ alkadienyl, C₃-C₇ cycloalkyl, C₃-C₇ cycloheteroalkyl, aryl, and heteroaryl, said group being substituted with 0-3 R⁵ and 0-3 R⁹.

20. A compound according to claim 1, wherein Functional entity precursor is selected among the group consisting of C₁-C₃ alkylene-NR⁴₂, C₁-C₃ alkylene-NR⁴C(O)R⁸, C₁-C₃ alkylene-NR⁴C(O)OR⁸, C₁-C₂ alkylene-O—NR⁴₂, C₁-C₂ alkylene-O—NR⁴C(O)R⁸, and C₁-C₂ alkylene-O—NR⁴C(O)OR⁸ substituted with 0-3 R⁹.

21. A library of compounds according to claim 1, wherein each different member of the library comprises a complementing element having a unique sequence of nucleotides, which identifies the functional entity.

22. A method for transferring a functional entity to a recipient reactive group, comprising the steps of
providing one or more building blocks according to claim 1,
contacting the one or more building blocks with a corresponding encoding element associated with a recipient reactive group under conditions which allow for a recognition between the one or more complementing elements and the encoding elements, said contacting being performed prior to, simultaneously with, or subsequent to a transfer of the functional entity to the recipient reactive group.

23. The method according to claim 22, wherein the encoding element comprises one or more encoding sequences comprised of 1 to 50 nucleotides and the one or more complementing elements comprise a sequence of nucleotides complementary to one or more of the encoding sequences.

24. The method of 22, wherein the recipient reactive group is an amine group, which may be part of a chemical scaffold, and the linkage between the functional entity precursor and the scaffold is of the general chemical structure:
Scaffold-NH—X(═V)-Functional entity precursor
In which
X═—C—, —S—, —P—, —S(O)—, or —P(O)—, and
V═O, S, NH, or N—C₁-C₆ alkyl.

25. The method according to claim 24, wherein X is —C— and V is O.
